(19) United States
(12) Patent Application Publication
(10) Pub. No.: US 2019/0219579 A1
(43) Pub. Date: Jul. 18, 2019

(54) METHODS AND SYSTEMS FOR SCORING EXTRACELLULAR MATRIX BIOMARKERS IN TUMOR SAMPLES

(71) Applicant: Ventana Medical Systems, Inc., Tucson, AZ (US)

(72) Inventors: Sihem Khelifa, Oro Valley, AZ (US); Jie Pu, Oro Valley, AZ (US)

(21) Appl. No.: 16/359,923

(22) Filed: Mar. 20, 2019

Related U.S. Application Data
(63) Continuation of application No. PCT/EP2017/073846, filed on Sep. 21, 2017.
(60) Provisional application No. 62/398,787, filed on Sep. 23, 2016.

(51) Int. Cl. G01N 33/574; G16H 50/30; C12N 9/34; G06T 7/00; G01N 1/30

(52) U.S. Cl. CPC G01N 33/574; G16H 50/30; C12N 9/2428; G01N 2001/302; G01N 1/30; C12Y 302/01036; G06T 7/0012

(57) ABSTRACT

Methods and systems for scoring hyaluronan-stained tissue samples by assessing the area of tumor-associated extracellular matrix (ECM) with hyaluronan staining compared to the entire surface area of the relevant portion of the sample. The methods and systems of the present disclosure may be used to, for example, to select patients for receipt of specific treatments.

### CROSS-REFERENCE TO RELATED APPLICATIONS

This is a continuation of PCT/EP2017/073846, filed Sep. 21, 2017, which claims the benefit of United States Provisional Patent Application No. U.S. 62/398,787, filed Sep. 23, 2016, the content of each of which is incorporated herein by reference in its entirety.

### FIELD OF THE INVENTION

The present invention relates to methods and systems for scoring extracellular matrix (ECM) biomarkers (such as hyaluronan), in tissue samples, and their use in diagnosing and/or prognosing disease and/or predicting disease response to ECM-directed therapies.

### BACKGROUND OF THE INVENTION

Many ECM components have been implicated in disease pathogenesis. See Jarvelainen et al., Pharmacol Rev., Vol. 61, Issue 2, pp. 198-223 (2009). One example is hyaluronan (HA). HA has been a matter of interest for its different roles in ovulation, embryogenesis, wound healing, and inflammatory diseases (Meyer and Palmer, 1934, J Biol Chem 107:629-34; Dicker et al., 2014, Acta Biomater 10(4):1558-1570). Recently, HA has been identified in tumor microenvironments, which has made it an attractive target in cancer therapy (Kultti et al, 2012, Cancers 4(3):873-903). HA accumulates in many solid tumors and actively interacts with its binding molecules, the hyaladherins, which consist of extracelluar matrix (ECM) proteoglycans and glycoproteins (e.g., versican) and cell surface receptors (e.g., CD44, RHAMM) (Tammi et al., Semin Cancer Biol 18:288-295; Itano and Kimata, 2008, Semin Cancer Biol 18:268-274; Simpson and Lokeshwar, 2008, Front Biosci 13:5664-5680; Toole and Slomiany, 2008, Drug Resist Updat 11:110-121; Maxwell et al., J Cell Sci 121:925-932). These complex interactions play a role in tumor cell adhesion, motility, proliferation and invasion (Toole, 2004, Nat Rev Cancer 4:528-539). Further, there is growing evidence that abnormal HA accumulation in tumors hampers the therapeutic effect of standard cancer treatments.

Interestingly, enzymatic depletion of HA can induce a reorganization of the tumor microenvironment that can significantly impact tumor behavior. Multiple publications describe preclinical studies where intravenous administration of a pegylated hyaluronidase PH20 (PEGPH20) has been associated with decreased tissue interstitial fluid pressure (tIFP), expansion of tumor blood vessels, increased delivery of chemotherapeutics, tumor growth suppression and improved survival (Toole, 2009, Clin Cancer Res 15:7462-7468; Jiang et al, 2012, Anticancer Res 32:1203-1212). Thus, some cancers exhibiting abnormal HA accumulation (e.g., some pancreatic adenocarcinomas) may benefit from HA depletion.

Using HA as a prognostic factor, previous attempts at determining HA content in tumors have not been able to reproducibly and accurately identify the patients with tumors that would likely benefit from HA depletion.

For example, others have used stains to assess HA that were in general heterogeneous, not very specific, and/or lacked purity (see Jadin et al, 2014, Journal of Histochemistry & Cytochemistry 62(9):672-83; Tengblad A, 1979, Biochim Biophys Acta 578:281-289; Tammi et al, 1988, J Invest Dermatol 90:412-414; Wells et al., 1991, Acta Derm Venereol 71:232-238; Lindqvist et al, 1992, Clin Chem 38:127-132; Lin et al, 1997, J Histochem Cytochem 45:1157-1163; Kobayashi et al, 1999, Cell Tissue Res 296:587-597; Sakai Set al, 2000, J Invest Dermatol 114:1184-1187; Baier et al, 2007, Matrix Biol 26:348-358; Clark et al, 2011, Invest Ophthalmol Vis Sci 52:6511-6521). Others targeted HA associated to its synthetic (HAS1,2, and 3) or lytic enzymes (HYAL1-5 and PH20) and/or to its most common receptors (CD44 and RHAMM) (see Provenzano et al, 2012, Cancer Cell 21:418-429; Jiang et al. 2012, Anticancer Res. 32:1203-1212; Jacobson et al., 2003, Biochem. Biophys. Res. Commun. 305:1017-1023; Wilkinson et al, 2006, J. Cell. Physiol. 2006; Kim et al, 2004, Cancer Res. 64:4569-4576; Udabage et al., 2005, Exp. Cell Res. 310:205-217).

Other methods of HA assessment were not clearly defined and mainly qualitative. Some authors have cited “HA high” versus “HA low” tumors with no definite score or convincing histopathology imaging. HA staining intensities were used as parameters of classification as 1+(low HA) to (3+ high HA). No publication had cited a 2+ intermediate HA (see Provenzano et al, 2012, Cancer Cell 21:418-429; Kultti et al, 2014, Biomed Res Int. Article ID 817613; Hautmann et al., 2001, Journal of Urology 165:2068-2074; Whatcott et al., 2015, Clin Cancer Res 21(5) 3561-3568; de la Motte and Drazba, 2011, J Histochem and Cytochem 59(3):252-257).

Quantitative scoring methods were mainly done through photomicrography and digital image analysis, which made the assessment of HA more complicated. Despite pathologist-annotation of the stained slides, it was difficult to rely on a computerized system to differentiate the non-tumor-associated HA expression from the tumor associate HA expression (see Tool, 2009, Clin Cancer Res 15:7492-7468; Provenzano et al, 2012, Cancer Cell 21:418-429; Lokeshwar et al, 2005, Cancer Res. 65:7782-7789; Kultti et al, 2014, Biomed Res Int. Article ID 817613; see also U.S. Pat. No. 8,846,034; U.S. Pat. App. No. 2014/0348817). Those methods proved not only to be discordant with clinical outcome data but not reproducible by conventional glass slides examination of stained tissue samples by pathologists.

### SUMMARY OF THE INVENTION

Inventors have surprisingly discovered a method for assessing HA content in tumor samples. The method features assessing HA content in the extracellular matrix (ECM), relative to the entire tumor surface. Note that the methods of the aforementioned references all appear to relate to total HA staining. None of the cited references suggest relying on HA content in the ECM.

Without wishing to limit the present invention to any theory or mechanism, it is believed that the methods of the present invention assess HA content in the most pertinent area of action of HA in tumors. Indeed, HA exerts its most harmful effect on a tumor by accumulating in its ECM and by crosslinking to other matrix proteins (see Jadin et al, 2014, Journal of Histochemistry & Cytochemistry 62(9):672-83; Kultti et al, 2012, Cancers 4(3), 873-903).

The present invention also features companion diagnostics for helping to identify a patient with a particular tumor type (e.g., pancreatic ductal adenocarcinomas (PDA), breast cancer, non-small cell lung cancer (NSCLC), etc.) that may benefit from a particular therapy, e.g., HA therapy (e.g., PEGPH20, see Lokeshwar et al, 2005, Cancer Res. 65:7782-7789) in combination with standard of care therapy. The companion diagnostics of the present invention utilize the aforementioned ECM-based scoring methods for assessing HA content. Note that since PEGPH20 has no confirmed direct action on the tumor cell compartment, it is believed that the inconsistent expression of HA staining in tumor cells could be a distractor if added to the ECM-based scoring method of HA in a tumor when it comes to predict PEGPH20 effect. Thus, the methods of the present invention assess HA in the most pertinent area of action for PEGPH20-based enzymatic depletion of HA.

The ECM-based HA scoring methods of the present invention are supported by clinical outcome data. In comparison to HA low patients, HA high patients identified based on the scoring algorithm of the present invention have demonstrated greater treatment benefit from HA targeted therapy than from the standard of care alone.

Further, the ECM-based HA scoring methods of the present invention have been proven to be reproducible, trainable, and transferrable to the general pathology practice by reader precision studies and multiple reader training tests.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows examples of solid tumors overexpressing hyaluronan (HA).

FIG. 2 shows a schematic view of an HA staining workflow for a particular specimen.

FIG. 3A shows an example of acceptable HA staining showing high HA content.

FIG. 3B shows an example of acceptable HA staining showing low HA content.

FIG. 4A shows low HA status.

FIG. 4B shows high HA status.

FIG. 5 illustrates an exemplary HA scoring systems as disclosed herein.

FIG. 6A illustrates an exemplary workflow implemented on an image analysis system as disclosed herein, wherein the object identification function is executed on the whole image before the ROI generator function is executed.

FIG. 6B illustrates an exemplary workflow implemented on an image analysis system as disclosed herein, wherein the object identification function is executed only the ROI after the ROI generator function is executed.

FIG. 7 illustrates an exemplary computing system that may form part of an HA scoring system as disclosed herein.

### DETAILED DESCRIPTION OF THE INVENTION

The present disclosure features methods and systems for assessing or scoring content of ECM-related molecules in tissue samples, e.g., tumor samples.

### I. Definitions

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, or a combination thereof.

As used herein, the term “biomarker” shall refer to any molecule or group of molecules found in a biological sample that can be used to characterize the biological sample or a subject from which the biological sample is obtained. For example, a biomarker may be a molecule or group of molecules whose presence, absence, or relative abundance is: characteristic of a particular disease state; indicative of the severity of a disease or the likelihood of disease progression or regression; and/or predictive that a particular disease state will respond to a particular treatment.

As another example, the biomarker may be an infectious agent (such as a bacterium, fungus, virus, or other microorganism), or a substituent molecule or group of molecules thereof.

As used herein, the terms “sample” and “biological sample” shall refer to any composition obtained from a subject containing or suspected of containing a biomarker. The term includes purified or separated components of cells, tissues, or blood, e.g., DNA, RNA, proteins, cell-free portions, or cell lysates. The sample can be a formalin-fixed, paraffin-embedded (FFPE) tissue sample, e.g., from a tumor or metastatic lesion, e.g., primary tumor or metastatic tumor. The sample can also be from previously frozen or fresh tissue, or from a liquid sample, e.g., blood or a blood component (plasma or serum), urine, semen, saliva, sputum, mucus, semen, tear, lymph, cerebral spinal fluid, material washed from a swab, etc. Samples also may include constituents and components of in vitro cultures of cells obtained from an individual, including cell lines. The sample can also be partially processed from a sample directly obtained from an individual, e.g., cell lysate or blood depleted of red blood cells.

As used herein, the term “cellular sample” refers to any biological sample containing intact cells, such as cell cultures, bodily fluid samples or surgical specimens taken for pathological, histological, or cytological interpretation.

As used herein, the term “tissue sample” shall refer to a cellular sample that preserves the spatial relationship between the cells as they existed within the subject from which the sample was obtained. “Tissue sample” shall encompass both primary tissue samples (i.e. cells and tissues produced by the subject) and xenografts (i.e. foreign cellular samples implanted into a subject).

As used herein, “histochemical detection” refers to a process involving labeling a biomarker or other structures in a tissue sample with detection reagents in a manner that permits detection of the biomarker or other structures in the context of the spatial relationship between the structures of the tissue sample. Examples include affinity histochemistry (AHC), immunohistochemistry (IHC), chromogenic in situ hybridization (CISH), fluorescent in situ hybridization (FISH), silver in situ hybridization (SISH), and hematoxylin and eosin (H&E) staining of formalin-fixed, paraffin-embedded tissue sections.

As used herein, the term “section” shall refer to a thin slice of a tissue sample suitable for microscopic analysis, typically cut using a microtome. As an example, a section may be 4 to 5 microns thick. The present disclosure is not limited to 4 to 5 microns.

As used herein, the term “serial section” shall refer to any one of a series of sections cut in sequence from a tissue sample. For two sections to be considered “serial sections” of one another, they do not necessarily need to consecutive sections from the tissue, but they should generally contain the same tissue structures in the same cross-sectional relationship, such that the structures can be matched to one another after histological staining.

As used herein, the phrase “specific binding,” “specifically binds to,” or “specific for” refers to measurable and reproducible interactions such as binding between a target and a biomarker-specific agent, which is determinative of the presence of the target in the presence of a heterogeneous population of molecules including biological molecules. For example, a binding entity that specifically binds to a target is an antibody that binds this target with greater affinity, avidity, more readily, and/or with greater duration than it binds to other targets. In one embodiment, the extent of binding of a binding entity to an unrelated target is less than about 10% of the binding of the antibody to the target as measured, e.g., by a radioimmunoassay (RIA). In certain embodiments, a binding entity that specifically binds to a target has a dissociation constant (Kd) of ≤1 μM, ≤100 nM, ≤10 nM, ≤1 nM, or ≤0.1 nM. In another embodiment, specific binding can include, but does not require exclusive binding.

As used herein, the term “biomarker-specific agent” shall refer to any compound or composition that binds to a biomarker or a specific structure within that biomarker in a manner that permits a specific detection of the biomarker in a sample. Examples include: antibodies and antigen binding fragments thereof; and engineered specific binding structures, including ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DARPins (scaffold based on Ankyrin repeat proteins; Molecular Partners AG, Zürich, CH), ANTICALINs (scaffold based on lipocalins; Pieris A G, Freising, D E), NANOBODYs (scaffold based on VHH (camelid Ig); Ablynx N/V, Ghent, BE), TRANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, DK) (Descriptions of such engineered specific binding structures are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008), the content of which is incorporated by reference); and fusion proteins including at least a first domain capable of specifically binding to the biomarker (e.g. an antigen binding fragment of an antibody or a target-binding portion of a protein that binds to the biomarker) and a second portion that is adapted to facilitate binding of detection reagents to the fusion protein (e.g., a biotin label, an epitope tag, an Ig fragment, etc.).

A “detection reagent” when used in connection with a histochemical assay (including immunohistochemistry and affinity histochemistry) is any reagent that is used to deposit a stain in proximity to a biomarker-specific agent bound to a biomarker in a cellular sample. Non-limiting examples include secondary antibodies capable of binding to a biomarker-specific antibody; enzymes linked to such secondary antibodies; and chemicals reactive with such enzymes to effect deposition of a fluorescent or chromogenic stain; and the like.

When used as a noun, the term “stain” shall refer to any substance that can be used to visualize specific molecules or structures in a cellular sample for microscopic analysis, including bright field microscopy, fluorescent microscopy, electron microscopy, and the like. When used as a verb, the term “stain” shall refer to any process that results in deposition of a stain on a cellular sample (e.g., tissue sample, cytological sample, etc.).

The terms “individual”, “subject”, and “patient” are used interchangeably herein. The individual can be pre-diagnosis, post-diagnosis but pre-therapy, undergoing therapy, or post-therapy. In the context of the present disclosure, the individual is typically seeking medical care.

The term “obtaining a sample from an individual” means that a biological sample from the individual is provided for testing. The obtaining can be directly from the individual, or from a third party that directly obtained the sample from the individual.

The term “providing therapy for an individual” means that the therapy is prescribed, recommended, or made available to the individual. The therapy may be actually administered to the individual by a third party (e.g., an in-patient injection), or by the individual himself.

As used herein, a “tumor surface” shall refer to a portion of a tissue section characterized by one or more contiguous regions composed substantially entirely of invasive neoplastic cells and associated stroma.

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

### II. Histochemical Labeling and Detection Methods

In an embodiment, the scoring methodologies disclosed herein are performed on cellular samples that are histochemically stained for HA.

Histochemical staining is a technique that relies on deposition of a detectable agent in proximity to a biomarker of interest, such that detection of the detectable agent allows the distribution of the biomarker throughout a tissue section to be evaluated. Cytochemical staining is similar, except that cytological samples are used. Hereafter, when the term “histochemical” is used, it should be understood that both “histochemical” and “cytochemical” is intended, unless it is clear from the context that only “histochemical” is intended.

In an embodiment, the sample is histochemically stained for HA using an affinity histochemistry technique. In affinity histochemistry, the detectable agent is localized to the biomarker via binding of a biomarker-specific agent to the biomarker in the sample under conditions that promote specific binding between the biomarker-specific agent and the biomarker. Due to the inherent nature of different types of histological tissues composing the body as well as the complexity of target biomarkers, there are no universal “one-size-fits-all” staining protocols in affinity histochemistry. Rather, the target biomarker, sample type, detection reagent, and detection scheme are all taken in consideration and a foundational histochemical protocol is modified to suit the experimental needs. Generally, a workflow of histochemical staining is as follows:
a. Antigen Retrieval (if needed): the process of fixing tissues can result in “masking” of biomarkers (i.e., rendering the biomarker inaccessible to the detection reagent being used). To this end, samples are frequently subjected to “antigen retrieval methods” that allow the previously masked biomarker to be detected. Many antigen retrieval methods are known in the art, and multiple antigen retrieval methods often may be used for the same biomarker. See Shi et al., J. Histochemistry & Cytochemistry, Vol. 49, Issue 8, pp. 931-37 (2001); Tacha & Teixeira, J. Histotechnology, Vol. 25, Issue 4, pp. 237-42 (2002); O'Leary et al., Biotechnic & Histochemistry, Vol. 84, Issue 5, pp. 217-21 (2009);
b. Blocking: Tissue sections are treated with reagents to block endogenous sources of nonspecific staining such as enzymes, endogenous peroxidase, free aldehyde groups, immunoglobulins, and other irrelevant molecules that can mimic specific staining;
c. Permeabilization (if needed): Tissue sections are incubated with permeabilization buffer to facilitate penetration of antibodies and other staining reagents into the tissue;
d. Incubation with the biomarker-specific reagent;
e. Incubation with detection reagents if indirect methods are being used.
   In addition to these steps, wash steps may be performed in between each of these steps in order to remove residual reagents and to prevent reactivity of unused reagents from one step to interact with reagents from a subsequent step.

In an embodiment, the HA-specific reagent is a TNF-stimulated gene 6 (TSG-6)-based probe. TSG-6 is an ˜30 kDa secreted HA-binding glycoprotein encoded by Tumor necrosis factor-Stimulated Gene 6 expressed in many different types of cells and tissues in response to a wide variety of cytokines and growth factors (Milner and Day 2003). TSG-6 plays critical roles in the formation and remodeling of HA-rich extracellular matrices via HA-crosslinking during inflammatory and inflammation-like processes (Fulop et al. 2003; Milner et al. 2006; Selbi et al. 2006; Simpson et al. 2009). TSG-6 is composed of a 100 amino acid-long N-terminal HA-binding Link module and a C-terminal CUB (complement Clr/Cls, Uegf, Bmp1) module that binds to fibronectin (Lee et al. 1992; Kohda et al. 1996; Kuznetsova et al. 2008). The high affinity of the TSG-6 HA-binding Link module for HA (Kahmann et al. 2000; Lesley et al. 2002) made it a starting point to engineer a homogenous and specific reagent to detect HA. As used herein, a “TSG-6 based probe” shall refer to any polypeptide that: (1) contains a sufficient portion of a TSG-6 protein to facilitate specific binding to HA in a human tissue section; and (2) contains at least one structure that can facilitate deposition of detection reagents on the tissue sample.

In some embodiments, the detectable moiety is directly conjugated to the HA-specific reagent, and thus is deposited on the sample upon binding of the HA-specific reagent to its target (generally referred to as a direct labeling method). Direct labeling methods are often more directly quantifiable, but often suffer from a lack of sensitivity. In other embodiments, deposition of the detectable moiety is effected by the use of a detection reagent associated with the HA-specific reagent (generally referred to as an indirect labeling method). Indirect labeling methods have the increase the number of detectable moieties that can be deposited in proximity to the HA-specific reagent, and thus are often more sensitive than direct labeling methods, particularly when used in combination with dyes.

Detection schemes for affinity histochemistry are typically divided into “direct” and “indirect” methods. Direct detection is the fastest and shortest IHC protocol, requiring incubation of tissue sections with only a primary antibody conjugated to the fluorophore of choice. Direct detection may be better suited for the detection of strong, highly expressed tissue antigens. For example, direct detection may be the technique of choice when, due to the host species of the primary antibodies and the histological nature of tissue, use of secondary detection antibodies may cause strong nonspecific staining. Indirect detection typically is more sensitive than direct. The higher sensitivity of indirect detection is the result of the possibility of two secondary antibodies labeled with fluorophores interacting with a single molecule of primary antibody bound to its tissue target. Indirect detection allows for the ability to choose secondary antibodies with fluorophores of different colors, Stokes shifts, quantum yield, and fade resistance.

In one embodiment, the TSG-6-based probe is a fusion protein with an Fc region of an antibody (such as a goat Fc, rabbit Fc, mouse Fc, or rat Fc). Exemplary TSG6 fusions for use as a HA-specific detection reagent are disclosed at Jadin et al., J. Histological Cytochem., Vol. 62, Issue 9, pp. 672-83 (2014), the content of which is incorporated herein by reference in its entirety. The Fc region allows traditional secondary antibodies to be used as detection reagents to facilitate deposition of dyes. In a specific embodiment, the HA specific detection reagent is a TSG6-rabbit Fc fusion. The TSG6-Fc fusion facilitates detection of HA by mediating deposition of a detectable moiety in close proximity to the TSG6-Fc fusion when the TSG6-Fc fusion is bound to HA in the tissue section.

In some embodiments, an indirect method is used with a TSG6-Fc fusion, wherein the detectable moiety is deposited via an enzymatic reaction localized to the TSG6-Fc fusion when bound to the tissue section. Suitable enzymes for such reactions are well-known and include, but are not limited to, oxidoreductases, hydrolases, and peroxidases. Specific enzymes explicitly included are horseradish peroxidase (HRP), alkaline phosphatase (AP), acid phosphatase, glucose oxidase, β-galactosidase, β-glucuronidase, and β-lactamase. The enzyme may be directly conjugated to the TSG6-based HA-specific reagent, or may be indirectly associated with the TSG6-based HA-specific reagent via a labeling conjugate. As used herein, a “labeling conjugate” comprises:
(a) a specific detection reagent; and
(b) an enzyme conjugated to the specific detection reagent, wherein the enzyme is reactive with the chromogenic substrate, signaling conjugate, or enzyme-reactive dye under appropriate reaction conditions to effect in situ generation of the dye and/or deposition of the dye on the tissue sample.
   In non-limiting examples, the specific detection reagent of the labeling conjugate may be a secondary detection reagent (such as a species-specific secondary antibody capable of specifically binding to the Fc region of the TSG6-Fc fusion), a tertiary detection reagent (such as a species-specific tertiary antibody specific for a secondary antibody bound to the TSG6-Fc fusion, an anti-hapten antibody specific for a hapten-conjugated secondary antibody bound to the TSG6-Fc fusion, or a biotin-binding protein capable of binding to a biotinylated secondary antibody bound to the TSG6-Fc fusion), or other such arrangements. An enzyme thus localized to the sample-bound TSG6-Fc fusion can then be used in a number of schemes to deposit a detectable moiety.

In some cases, the enzyme reacts with a chromogenic compound/substrate. Particular non-limiting examples of chromogenic compounds/substrates include 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate](ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, or tetrazolium violet.

In some embodiments, the enzyme can be used in a metallographic detection scheme. Metallographic detection methods include using an enzyme such as alkaline phosphatase in combination with a water-soluble metal ion and a redox-inactive substrate of the enzyme. In some embodiments, the substrate is converted to a redox-active agent by the enzyme, and the redox-active agent reduces the metal ion, causing it to form a detectable precipitate. (see, for example, U.S. patent application Ser. No. 11/015,646, filed Dec. 20, 2004, PCT Publication No. 2005/003777 and U.S. Patent Application Publication No. 2004/0265922; each of which is incorporated by reference herein in its entirety). Metallographic detection methods include using an oxido-reductase enzyme (such as horseradish peroxidase) along with a water soluble metal ion, an oxidizing agent and a reducing agent, again to for form a detectable precipitate. (See, for example, U.S. Pat. No. 6,670,113, which is incorporated by reference herein in its entirety).

In some embodiments, the enzymatic action occurs between the enzyme and the dye itself, wherein the reaction converts the dye from a non-binding species to a species deposited on the sample. For example, reaction of DAB with a peroxidase (such as horseradish peroxidase) oxidizes the DAB, causing it to precipitate.

In yet other embodiments, the detectable moiety is deposited via a signaling conjugate comprising a latent reactive moiety configured to react with the enzyme to form a reactive species that can bind to the sample or to other detection components. These reactive species are capable of reacting with the sample proximal to their generation, i.e. near the enzyme, but rapidly convert to a non-reactive species so that the signaling conjugate is not deposited at sites distal from the site at which the enzyme is deposited. Examples of latent reactive moieties include: quinone methide (QM) analogs, such as those described at WO2015124703A1, and tyramide conjugates, such as those described at, WO2012003476A2, each of which is hereby incorporated by reference herein in its entirety. In some examples, the latent reactive moiety is directly conjugated to a dye, such as N,N′-biscarboxypentyl-5,5′-disulfonato-indo-dicarbocyanine (Cy5), 4-(dimethylamino) azobenzene-4′-sulfonamide (DABSYL), tetramethylrhodamine (DISCO Purple), and Rhodamine 110 (Rhodamine) In other examples, the latent reactive moiety is conjugated to one member of a specific binding pair, and the dye is linked to the other member of the specific binding pair. In other examples, the latent reactive moiety is linked to one member of a specific binding pair, and an enzyme is linked to the other member of the specific binding pair, wherein the enzyme is (a) reactive with a chromogenic substrate to effect generation of the dye, or (b) reactive with a dye to effect deposition of the dye (such as DAB). Examples of specific binding pairs include:
(1) a biotin or a biotin derivative (such as desthiobiotin) linked to the latent reactive moiety, and a biotin-binding entity (such as avidin, streptavidin, deglycosylated avidin (such as NEUTRAVIDIN), or a biotin binding protein having a nitrated tyrosine at its biotin binding site (such as CAPTAVIDIN) linked to a dye or to an enzyme reactive with a chromogenic substrate or reactive with a dye (for example, a peroxidase linked to the biotin-binding protein when the dye is DAB);
(2) a hapten linked to the latent reactive moiety, and an anti-hapten antibody linked to a dye or to an enzyme reactive with a chromogenic substrate or reactive with a dye (for example, a peroxidase linked to the biotin-binding protein when the dye is DAB).

Non-limiting examples of TSG6-Fc fusion and detection reagent combinations are set forth in Table 1 are specifically included.

TABLE 1
A. Secondary detection reagent linked directly to detectable moiety
TSG6-Fc fusion + 2° Fc-specific detection reagent-Dye conjugate
B. Secondary detection reagent linked to Enzyme reacting with
detectable moiety
TSG6-Fc fusion + 2° Fc-specific detection reagent-Enzyme conjugate + DAB
TSG6-Fc fusion + 2° Fc-specific detection reagent-Enzyme conjugate +
Chromogen
C. Secondary detection reagent linked to Enzyme reacting with
detectable moiety

| | |
|---|---|
| C1. Signaling conjugate | TSG6-Fc fusion + 2° Fc-specific detection reagent- |
| comprises detectable moiety | Enzyme conjugate + QM-Dye conjugate |
| | TSG6-Fc fusion + 2° Fc-specific detection reagent- |
| | Enzyme conjugate + Tyramide-Dye |
| | conjugate |
| C2. Signaling conjugate comprises | TSG6-Fc fusion + 2° specific detection reagent- |
| enzyme thatr eacts directly with | Enzyme conjugate + QM-Enzyme conjugate + |
| detectable moiety | DAB |
| | TSG6-Fc fusion + 2° Fc-specific detection reagent- |
| | Enzyme conjugate + QM-Enzyme conjugate + |
| | Chromogen |
| | TSG6-Fc fusion + 2° Fc-specific detection reagent- |
| | Enzyme conjugate + Tyramide-Enzyme |
| | conjugate + DAB |
| | TSG6-Fc fusion + 2° Fc-specific detection reagent- |
| | Enzyme conjugate + Tyramide-Enzyme |
| | conjugate + Chromogen |
| C3. Signaling | TSG6-Fc fusion + 2° Fc-specific detection reagent- |
| conjugate comprises | Enzyme conjugate + QM-Enzyme conjugate + |
| enzyme that reacts with | QM-Dye conjugate |
| second signaling conjugate | TSG6-Fc fusion + 2° Fc-specific detection reagent- |
| comprising detectable moiety | Enzyme conjugate + QM-Enzyme conjugate + |
| | Tyramide-Dye conjugate |
| | TSG6-Fc fusion + 2° Fc-specific detection reagent- |
| | Enzyme conjugate + Tyramide-Enzyme |
| | conjugate + QM-Dye conjugate |
| | TSG6-Fc fusion + 2° Fc-specific detection reagent- |
| | Enzyme conjugate + Tyramide-Enzyme |
| | conjugate + Tyramide-Dye conjugate |
| C4. Signaling | TSG6-Fc fusion + 2° Fc-specific detection reagent- |
| conjugate comprises | Enzyme conjugate + Tyramide- |
| member of a specific | (biotin/hapten) conjugate + Dye-(avidin/anti- |
| binding pair and other | hapten) conjugate |
| member of binding pair is | TSG6-Fc fusion + 2° Fc-specific detection reagent- |
| linked to detectable | Enzyme conjugate + QM-(biotin/hapten) |
| moiety | conjugate + Dye-(avidin/anti-hapten) |
| | conjugate |
| C5. Signaling conjugate | TSG6-Fc fusion + 2° Fc-specific detection reagent- |
| comprises member of a | Enzyme conjugate + QM-(biotin/hapten) |
| specific binding pair and | conjugate + Enzyme-(avidin/anti-hapten) |
| other member of binding | conjugate + DAB |
| pair is linked to enzyme | TSG6-Fc fusion + 2° Fc-specific detection reagent- |
| reactive with detectable moiety | Enzyme conjugate + QM-(biotin/hapten) |
| | conjugate + Enzyme-(avidin/anti-hapten) |
| | conjugate + Chromogen |
| | TSG6-Fc fusion + 2° Fc-specific detection reagent- |
| | Enzyme conjugate + Tyramide- |
| | (biotin/hapten) conjugate + Enzyme- |
| | (avidin/anti-hapten) conjugate + DAB |
| | TSG6-Fc fusion + 2° Fc-specific detection reagent- |
| | Enzyme conjugate + Tyramide- |
| | (biotin/hapten) conjugate + Enzyme- |
| | (avidin/anti-hapten) conjugate + Chromogen |
| C6. Signaling conjugate | TSG6-Fc fusion + 2° Fc-specific detection reagent- |
| comprises member of a | Enzyme conjugate + QM-(biotin/hapten) |
| specific binding pair and | conjugate + Enzyme-(avidin/anti-hapten) |
| other member of binding | conjugate + Tyramide-Dye conjugate |
| pair is linked to enzyme | TSG6-Fc fusion + 2° Fc-specific detection reagent- |
| reactive with second detectable | Enzyme conjugate + QM-(biotin/hapten) |
| moiety linked to a detectable | conjugate + Enzyme-(avidin/anti-hapten) |
| moiety | conjugate + QM-Dye conjugate |
| | TSG6-Fc fusion + 2° Fc-specific detection reagent- |
| | Enzyme conjugate + Tyramide- |
| | (biotin/hapten) conjugate + Enzyme- |
| | (avidin/anti-hapten) conjugate + Tyramide- |
| | Dye conjugate |
| | TSG6-Fc fusion + 2° Fc-specific detection reagent- |
| | Enzyme conjugate + Tyramide- |
| | (biotin/hapten) conjugate + Enzyme- |
| | (avidin/anti-hapten) conjugate + QM-Dye |
| | conjugate |

D. Secondary detection reagent linked to member of specific binding
pair

| | |
|---|---|
| D1. Dye linked to other | TSG6-Fc fusion + 2° Fc-specific detection reagent- |
| member of specific binding pair | (biotin/hapten) conjugate + Dye-(avidin/anti- |
| | hapten) conjugate |
| D2. Enzyme linked to other | TSG6-Fc fusion + 2° Fc-specific detection reagent- |
| member of specific binding | (biotin/hapten) conjugate + Enzyme- |
| pair, wherein the enzyme is | (avidin/anti-hapten) conjugate + DAB |
| reactive with detectable moiety | TSG6-Fc fusion + 2° Fc-specific detection reagent- |
| | (biotin/hapten) conjugate + Enzyme- |
| | (avidin/anti-hapten) conjugate + Chromogen |
| | TSG6-Fc fusion + 2° Fc-specific detection reagent- |
| | (biotin/hapten) conjugate + Enzyme- |
| | (avidin/anti-hapten) conjugate + QM-Dye |
| | conjugate |
| | TSG6-Fc fusion + 2° Fc-specific detection reagent- |
| | (biotin/hapten) conjugate + Enzyme- |
| | (avidin/anti-hapten) conjugate + Tyramide- |
| | Dye conjugate |

E. Tertiary specific detection reagent linked directly to detectable
moiety
TSG6-Fc fusion + 2° Fc-specific detection reagent + 3° specific detection
reagent-Dye conjugate
F. Tertiary specific detection reagent linked to Enzyme reacting with
detectable moiety
TSG6-Fc fusion + 2° Fc-specific detection reagent + 3° specific detection
reagent-Enzyme conjugate + DAB
TSG6-Fc fusion + 2° Fc-specific detection reagent + 3° specific detection
reagent-Enzyme conjugate + Chromogen
G. Tertiary specific detection reagent linked to Enzyme reacting with
detectable moiety

| | |
|---|---|
| G1. Signaling conjugate | TSG6-Fc fusion + 2° Fc-specific detection reagent + |
| comprises detectable moiety | 3° specific detection reagent-Enzyme |
| | conjugate + QM-Dye conjugate |
| | TSG6-Fc fusion + 2° Fc-specific detection reagent + |
| | 3° specific detection reagent-Enzyme |
| | conjugate + Tyramide-Dye conjugate |
| G2. Signaling conjugate | TSG6-Fc fusion + 2° Fc-specific detection reagent + |
| comprises enzyme that | 3° specific detection reagent-Enzyme |
| reacts directly with detectable | conjugate + QM-Enzyme conjugate + DAB |
| moiety | TSG6-Fc fusion + 2° Fc-specific detection reagent + |
| | 3° specific detection reagent-Enzyme |
| | conjugate + QM-Enzyme conjugate + |
| | Chromogen |
| | TSG6-Fc fusion + 2° Fc-specific detection reagent + |
| | 3° specific detection reagent-Enzyme |
| | conjugate + Tyramide-Enzyme conjugate + |
| | DAB |
| | TSG6-Fc fusion + 2° Fc-specific detection reagent + |
| | 3° specific detection reagent-Enzyme |
| | conjugate + Tyramide-Enzyme conjugate + |
| | Chromogen |
| G3. Signaling | TSG6-Fc fusion + 2° Fc-specific detection reagent + |
| conjugate comprises | 3° specific detection reagent-Enzyme |
| enzyme that reacts with | conjugate + QM-Enzyme conjugate + QM- |
| second signaling | Dye conjugate |
| conjugate comprising | TSG6-Fc fusion + 2° Fc-specific detection reagent + |
| detectable moiety | 3° specific detection reagent-Enzyme |
| | conjugate + QM-Enzyme conjugate + |
| | Tyramide-Dye conjugate |
| | TSG6-Fc fusion + 2° Fc-specific detection reagent + |
| | 3° specific detection reagent-Enzyme |
| | conjugate + Tyramide-Enzyme conjugate + |
| | QM-Dye conjugate |
| | TSG6-Fc fusion + 2° Fc-specific detection reagent + |
| | 3° specific detection reagent-Enzyme |
| | conjugate + Tyramide-Enzyme conjugate + |
| | Tyramide-Dye conjugate |
| G4. Signaling | TSG6-Fc fusion + 2° Fc-specific detection reagent + |
| conjugate comprises | 3° specific detection reagent-Enzyme |
| member of a specific | conjugate + Tyramide-(biotin/hapten) |
| binding pair and other | conjugate + Dye-(avidin/anti-hapten) |
| member of binding pair is | conjugate |
| linked to detectable | TSG6-Fc fusion + 2° Fc-specific detection reagent + |
| moiety | 3° specific detection reagent-Enzyme |
| | conjugate + QM-(biotin/hapten) conjugate + |
| | Dye-(avidin/anti-hapten) conjugate |
| G5. Signaling | TSG6-Fc fusion + 2° Fc-specific detection reagent + |
| conjugate comprises | 3° specific detection reagent-Enzyme |
| member of a specific | conjugate + QM-(biotin/hapten) conjugate + |
| binding pair and other | Enzyme-(avidin/anti-hapten) conjugate + |
| member of binding pair is | DAB |
| linked to enzyme reactive | TSG6-Fc fusion + 2° Fc-specific detection reagent + |
| with detectable moiety | 3° specific detection reagent-Enzyme |
| | conjugate + QM-(biotin/hapten) conjugate + |
| | Enzyme-(avidin/anti-hapten) conjugate + |
| | Chromogen |
| | TSG6-Fc fusion + 2° Fc-specific detection reagent + |
| | 3° specific detection reagent-Enzyme |
| | conjugate + Tyramide-(biotin/hapten) |
| | conjugate + Enzyme-(avidin/anti-hapten) |
| | conjugate + DAB |
| | TSG6-Fc fusion + 2° Fc-specific detection reagent + |
| | 3° specific detection reagent-Enzyme |
| | conjugate + Tyramide-(biotin/hapten) |
| | conjugate + Enzyme-(avidin/anti-hapten) |
| | conjugate + Chromogen |
| G6. Signaling | TSG6-Fc fusion + 2° Fc-specific detection reagent + |
| conjugate comprises | 3° specific detection reagent-Enzyme |
| member of a specific | conjugate + QM-(biotin/hapten) conjugate + |
| binding pair and other | Enzyme-(avidin/anti-hapten) conjugate + |
| member of binding pair is | Tyramide-Dye conjugate |
| linked to enzyme reactive | TSG6-Fc fusion + 2° Fc-specific detection reagent + |
| with second detectable | 3° specific detection reagent-Enzyme |
| moiety linked to a detectable | conjugate + QM-(biotin/hapten) conjugate + |
| moiety | Enzyme-(avidin/anti-hapten) conjugate + |
| | QM-Dye conjugate |
| | TSG6-Fc fusion + 2° Fc-specific detection reagent + |
| | 3° specific detection reagent-Enzyme |
| | conjugate + Tyramide-(biotin/hapten) |
| | conjugate + Enzyme-(avidin/anti-hapten) |
| | conjugate + Tyramide-Dye conjugate |
| | TSG6-Fc fusion + 2° Fc-specific detection reagent + |
| | 3° specific detection reagent-Enzyme |
| | conjugate + Tyramide-(biotin/hapten) |
| | conjugate + Enzyme-(avidin/anti-hapten) |
| | conjugate + QM-Dye conjugate |

H. Tertiary specific detection reagent linked to member of specific
binding pair

| | |
|---|---|
| H1. Dye linked to | TSG6-Fc fusion + 2° Fc-specific detection reagent + |
| other member of specific | 3° specific detection reagent-(biotin/hapten) |
| binding pair | conjugate + Dye-(avidin/anti-hapten) |
| | conjugate |
| H2. Enzyme linked to other | TSG6-Fc fusion + 2° Fc-specific detection reagent + |
| member of specific binding | 3° specific detection reagent-(biotin/hapten) |
| pair, wherein the enzyme is | conjugate + Enzyme-(avidin/anti-hapten) |
| reactive with detectable moiety | conjugate + DAB |
| | TSG6-Fc fusion + 2° Fc-specific detection reagent + |
| | 3° specific detection reagent-(biotin/hapten) |
| | conjugate + Enzyme-(avidin/anti-hapten) |
| | conjugate + Chromogen |
| | TSG6-Fc fusion + 2° Fc-specific detection reagent + |
| | 3° specific detection reagent-(biotin/hapten) |
| | conjugate + Enzyme-(avidin/anti-hapten) |
| | conjugate + QM-Dye conjugate |
| | TSG6-Fc fusion + 2° Fc-specific detection reagent + |
| | 3° specific detection reagent-(biotin/hapten) |
| | conjugate + Enzyme-(avidin/anti-hapten) |
| | conjugate + Tyramide-Dye conjugate |

In a specific embodiment, the 2° Fc-specific detection reagents set forth in Table 1 are antibodies. In another embodiment, the TSG6-Fc fusion is a TSG6-Rabbit Fc fusion, and the 2° Fc-specific detection reagent is an anti-rabbit Ig antibody.

Non-limiting examples of commercially available detection reagents or kits comprising detection reagents suitable for use with present methods include: VENTANA ultraView detection systems (secondary antibodies conjugated to enzymes, including HRP and AP); VENTANA iVIEW detection systems (biotinylated anti-species secondary antibodies and streptavidin-conjugated enzymes); VENTANA OptiView detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OptiView Amplification system (Anti-species secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OmniMap, DISCOVERY UltraMap anti-hapten antibody, secondary antibody, chromogen, fluorophore, and dye kits, each of which are available from Ventana Medical Systems, Inc. (Tucson, Ariz.); PowerVision and PowerVision+ IHC Detection Systems (secondary antibodies directly polymerized with HRP or AP into compact polymers bearing a high ratio of enzymes to antibodies); and DAKO EnVision™+ System (enzyme labeled polymer that is conjugated to secondary antibodies).

### III. Scoring Methods

The fundamental characteristics of a scoring system were suggested by Crissman et al., and included the following: (1) scoring system should be definable, (2) it should be reproducible, and (3) it should produce meaningful results. Gibson-Corley et al. also described some key principles for an appropriate scoring system and data evaluation: “Masking” of the experimental material to reduce the subjectivity of valued scores; a thorough “Examination” of all tissues/slides with creation of a context for scoring tissue lesions; specifying “Lesion parameters”, which then could be used as score categories; using a clear “Scoring definitions” will improve understanding of presented data and increase repeatability of scoring system; whenever possible, use “Interpretation Consistency” which imply that all the samples are scored by the same scientist in a reasonable period of time.

Semiquantitative scoring systems are widely used to convert subjective perception of IHC-marker expression by histopathologists into quantitative data, which is then used for statistical analyses and establishing of the conclusions. Without scoring system the description of received data can be provided only with subjective perception, expressed in such adjectives as “strong”, “weak”, “absent” with modifiers as “more” or “less”. Each pathologist uses this approach while examining the slides, but without conversion into a scoring system, they are just subjective expressions of assessments of solely one pathologist. To reduce subjectivity it is recommended to have at least more than one observer in the study.

Most semiquantitative scoring systems usually include multiple parameters that are separately quantified and finally combined in a total score. Scores of the different experimental groups can then be compared by statistical tests. The selection of the parameters may be based on the scientific hypothesis or question together with the morphological features of expression of IHC markers that are used in an experiment. The “golden standard” in standardized IHC scoring is defined for the evaluation of only 3 markers so far: Her2/neu, estrogen (ER), and progesterone (PgR) for which testing guidelines have been developed. For many IHC markers scientists design an individual scoring system.

The scoring and staining methods disclosed herein may be used for scoring ECM components. In an embodiment, the ECM component is a biomarker of a disease state. Exemplary ECM components useful as disease state biomarkers are disclosed at Jarvelainen et al., Pharmacol Rev., Vol. 61, Issue 2, pp. 198-223 (2009), the content of which is incorporated herein by reference in its entirety. In exemplary embodiments, the ECM component is used as a predictive biomarker for a human therapy, such as a companion diagnostic. In a particular embodiment, the scoring methods of the present disclosure may be used to score a particular ECM component as a predictive biomarker of a response to an ECM-modifying therapy.

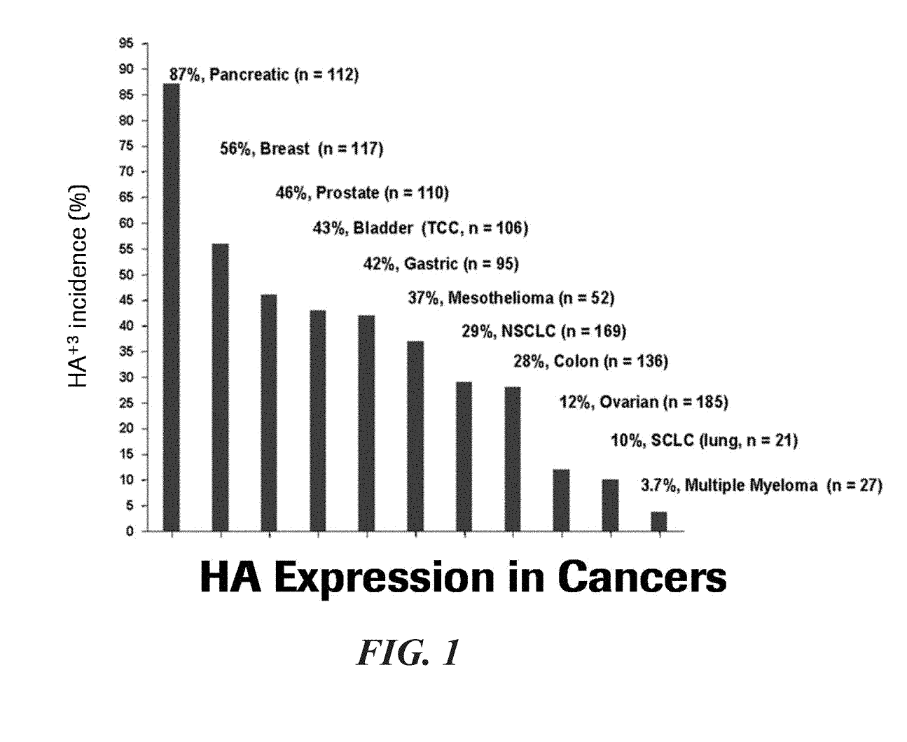

In some embodiments, the ECM-related molecule is hyaluronic acid (HA). It is estimated that approximately 20-30% of all solid tumors overexpress HA to varying degrees. FIG. 1 shows non-limiting examples of tumors that feature abnormal HA expression. The present disclosure also features scoring HA in a breast tumor, a prostate tumor, a bladder tumor, a lung tumor, a colon tumor, an ovarian tumor, etc.

In a specific embodiment, a scoring method of the present disclosure features assessing HA content (or other appropriate ECM biomarker content) in the extracellular matrix (ECM), relative to the entire tumor surface. The scoring method of the present disclosure comprises identifying the tumor surface (TS). Identifying the tumor surface involves identifying tumor cells and associated stroma (e.g., on the H&E slide). Note that organ capsule area and fibrotic pseudo-capsule area should not be included in the tumor surface, nor should necrotic areas. Non-tumor-associated structures entrapped in the tumor, such as muscle, collagen bundles, adipose tissue and nerves, might normally express HA. They are considered part of the tumor surface; however, HA expression within these non-tumor-associated structures is not scored. As used herein, “tumor-associated extracellular matrix” or “tumor-associated ECM” shall refer to ECM areas within the tumor surface that is not associated with non-tumor-associated structures entrapped in the tumor. The method may further comprise confirming that the negative control run (e.g., stained slide on a consecutive cut section) does not display non-specific moderate or strong background that may interfere with HA reading. One of ordinary skill in the art can determine the level of non-specific background (e.g., acceptable faint to weak diffuse and non-specific background that does not interfere with HA interpretation). The method further comprises estimating the percentage of HA-stained extracellular matrix (at any intensity level above background) over the entire tumor surface.

The scoring method of the present disclosure may comprise making HA visible in the tissue (e.g., tumor) sample, e.g., by staining the tissue sample for HA, and determining the area of the extracellular matrix (ECM) that has HA staining of any intensity over background (e.g., weak (1+) staining for HA, moderate (2+) staining for HA, or strong (3+) staining for HA) divided by the area of the entire tumor surface as a percentage to create an HA score. Formula 1 reflects the aforementioned method.

$$HA Score = ( \frac{area ( ECM )}{area ( TS )} \times 100 \% ) \qquad Formula 1$$

wherein area(ECM) is the area of the tumor-associated extracellular matrix having HA staining at any intensity above background and area(TS) is the total surface area of the tumor surface.

Referring to Formula 2 below, the HA score may be determined slightly differently compared to Formula 1. For example, in some embodiments, the HA score is calculated by totaling (1) the area of the tumor-associated ECM with weak (1+) staining for HA over the area of the tumor surface as a percentage; (2) the area of the tumor-associated ECM with moderate (2+) staining for HA over the area of the tumor surface as a percentage; and (3) the area of the tumor-associated ECM with strong (3+) staining for HA over the area of the tumor surface as a percentage. Formula 2 reflects this calculation.

$$HA Score = ( \frac{area ( ECM w / 1 + )}{area ( TS )} + \frac{area ( ECM w / 2 + )}{area ( TS )} + \frac{area ( ECM w / 3 + )}{area ( TS )} ) \times 100 \% \qquad Formula 2$$

wherein:
area(ECM w/1+) is the area of the tumor-associated extracellular matrix having HA staining intensity of 1+;
area(ECM w/2+) is the area of the tumor-associated extracellular matrix having HA staining intensity of 2+;
area(ECM w/3+) is the area of the tumor-associated extracellular matrix having HA staining intensity of 3+; and
area(TS) is the total surface area of the tumor surface.
   When formula 2 is implemented on a system as set forth herein, “1+,” “2+,” and “3+” categories may be replaced by “low,” “medium,” and “high” HA staining intensities or other subdivisions of staining intensity as desired by the user.

While mathematically Formula 1 and Formula 2 calculate an identical HA score, the process by which an individual (e.g., pathologist) scores HA content using Formula 1 and Formula 2 is different, e.g., in Formula 1, the pathologist looks for any HA staining above background in the tumor-associated ECM, whereas in Formula 2, the pathologist first looks for weak HA staining in the tumor-associated ECM, then moderate HA staining in the tumor-associated ECM, and then strong HA staining in the tumor-associated ECM (though not necessarily in that order). The subtle differences in these processes may cause there to be a slightly different HA score if evaluated by one formula versus the other.

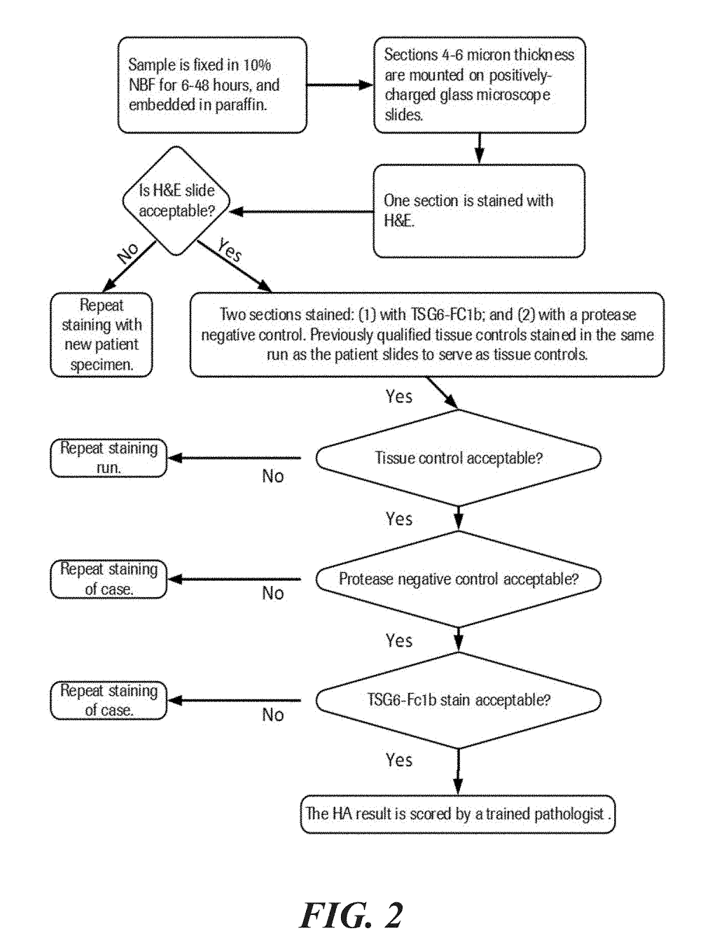

FIG. 2 shows a schematic view of the workflow for a particular specimen. As a non-limiting example, in some embodiments, a tissue sample is taken from a patient and fixed (e.g., in NFB or other appropriate system) and embedded in paraffin. Sections of the sample are mounted on microscope slides. One section (or more) is stained with H&E. If the H&E staining is acceptable, one section is stained for HA, and another section is stained for a negative control (e.g., stained with protease negative reagent control) in the same staining run. If necessary, other tissue controls (e.g., normal skin, normal liver, etc.) are stained in the same run as the patient slides to serve as tissue controls. If the tissue control is acceptable and the negative control acceptable, the HA slide is evaluated. If the HA slide is acceptable, the sample is evaluated by a pathologist using scoring methods according to the present disclosure.

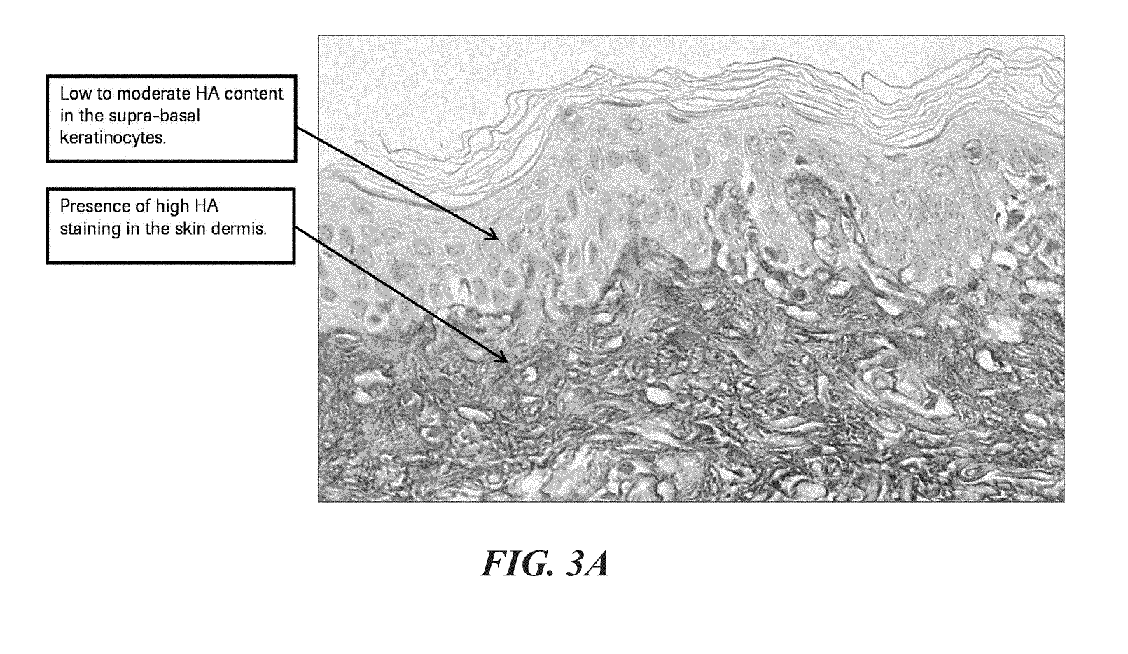

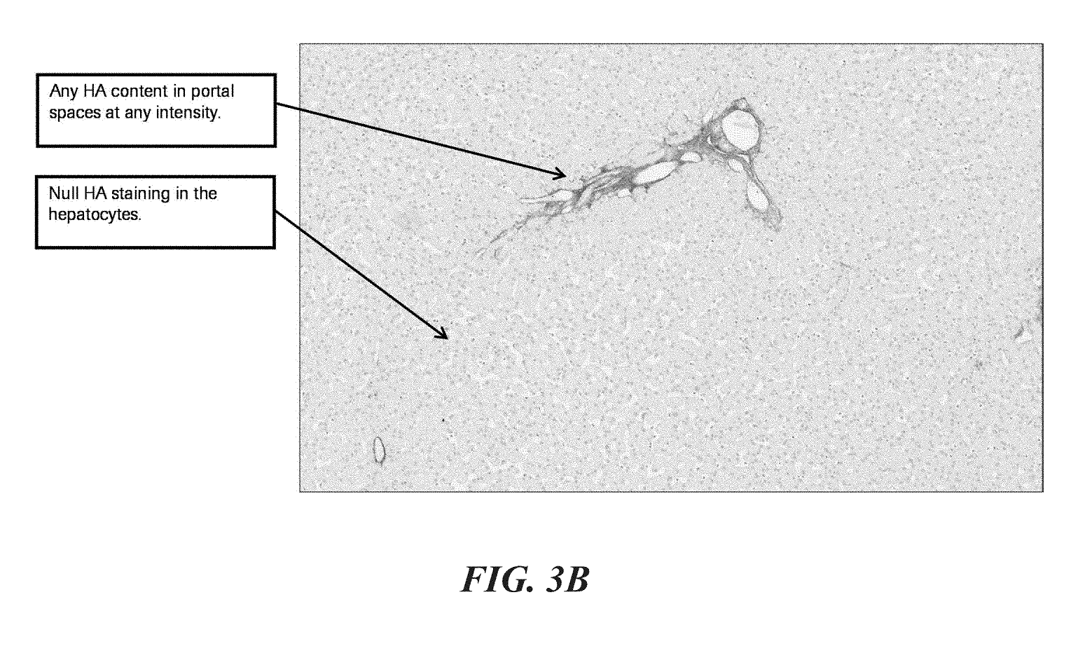

As an example of evaluation of a tissue control, for a liver control tissue, an acceptable stain would show null HA content in the hepatocytes and any HA content in portal spaces (at any intensity). In some embodiments, an unacceptable stain would show any HA content in the hepatocytes and/or excessive non-specific background staining of the liver tissue. FIG. 3A shows an example of acceptable HA staining showing high HA content: there is low to moderate HA content in the supra-basal keratinocytes and the presence of high HA staining in the skin dermis. FIG. 3B shows acceptable HA staining showing low HA content: there is HA content in portal spaces and null HA staining in the hepatocytes.

The HA score of a sample may be compared to a particular threshold value. For example, if the HA score of the sample is greater than (or equal to) a threshold value, the sample may be designated as having a high HA score. Or, if the HA score of the sample is less than (or in some cases equal to) the threshold value, the sample may be designated as having a low HA score. The threshold values may be determined using appropriate data such as clinical data. The threshold values may differ depending on the biomarker and/or the cancer type. In some embodiments, the threshold value is 50% (e.g., for pancreatic ductal adenocarcinoma). For example, a score of 50% or more (e.g., greater than or equal to 50%) in a particular tumor (e.g., pancreatic ductal adenocarcinoma) may be designated as high HA. In some embodiments, a score of less than 50% in a particular tumor (e.g., pancreatic ductal adenocarcinoma) may be designated as low HA. The present disclosure is not limited to these thresholds. For example, a score of greater than or equal to 25% may be designated as high HA, and a score of less than 25% may be designated as low HA. Or, a score of greater than or equal to 75% may be designated as high HA and a score of less than 75% may be designated as low HA. Further, these thresholds may be different for other tumor types. For example, an HA score of 25% or more may be considered high HA for a tumor type such as gastric cancer or lung cancer, whereas a score of greater than or equal to 50% may be considered high HA for a pancreatic tumor. Cutoffs or thresholds may be determined by technical assessment of scores acquired from cohorts or other appropriate means.

As previously discussed, the tumor-associated ECM-based HA scoring methods of the present disclosure are supported by clinical outcome data. In comparison to HA low patients, HA high patients identified based on the scoring algorithm of the present disclosure have demonstrated greater treatment benefit from HA targeted therapy than from the standard of care alone (see Table 2).

TABLE 2
Treatment Efficacy vs. HA Status

| | Treatment/Control Odds Ratio for | |
|---|---|---|
| HA Status | Objective Response Rate | 95% Confidence Limits |
| High | 9.17 | [1.49, 56.28] |
| Low | 1.00 | [0.32, 3.12] |

Further, the ECM-based HA scoring methods of the present disclosure have been proven to be reproducible, trainable, and transferrable to the general pathology practice by reader precision studies and multiple reader training tests (see Table 2, Table 3; OPA=Overall Percent Agreement; APA=Average Positive Agreement; ANA=Average Negative Agreement; [a]95% CI=2-sided 95% confidence interval calculated using the percentile bootstrap method from 5,000 bootstrap samples).

TABLE 3
Inter-Reader Precision

| | | Reader 2 | | |
|---|---|---|---|---|
| Reader Pair | Reader 1 | HA High | HA Low | Total |
| All Reader | HA High | 137 | 9 | 146 |
| Pairs | HA Low | 7 | 147 | 154 |
| | Total | 144 | 156 | 300 |
| | OPA n/N (%) (95% CI) | 284/300 | (90.7, 98.0) | |
| | [a] | (94.7) | | |
| | APA n/N (%) (95% CI) | 274/290 | (90.0, 98.0) | |
| | [a] | (94.5) | | |
| | ANA n/N (%) (95% CI) | 294/310 | (91.0, 98.1) | |
| | [a] | (94.8) | | |

TABLE 4
Intra-Reader Precision

| | | Round 2 | | |
|---|---|---|---|---|
| Reader | Round 1 | HA High | HA Low | Total |
| All Readers | HA High | 140 | 5 | 145 |
| | HA Low | 12 | 143 | 155 |
| | Total | 152 | 148 | 300 |
| | OPA n/N (%) (95% CI) | 283/300 | (91.3, 97.0) | |
| | [a] | (94.3) | | |
| | APA n/N (%) (95% CI) | 280/297 | (90.8, 97.1) | |
| | [a] | (94.3) | | |
| | ANA n/N (%) (95% CI) | 286/303 | (91.1, 97.1) | |
| | [a] | (94.4) | | |

The present disclosure also features predictive diagnostics for helping to identify a patient with a particular tumor type (e.g., breast tumor, lung tumor (including non-small cell lung cancer (NSCLC)), prostate tumor, pancreatic tumor (including pancreatic ductal adenocarcinoma (PDA)), gastrointestinal tumor, urogenital tumor, etc.) that may benefit from a particular therapy, e.g., HA therapy (e.g., PEGPH20). The predictive diagnostics of the present disclosure utilize the aforementioned ECM-based scoring methods for assessing HA content. A high HA score may be indicative of a patient who may benefit from HA therapy (e.g., PEGPH20), e.g., a patient who may more likely benefit from anti-HA therapy. Or, a low HA score may be indicative of a patient who may not benefit from HA therapy. Typically, the HA therapy is used to improve the efficacy of other anti-tumor therapeutic entities (such as a chemotherapeutics, radiation therapy, or a targeted therapeutic), and thus is typically co-administered with such anti-tumor therapeutic entities or is administered shortly before administration of other anti-tumor therapeutic entities.

### IV. HA Scoring Systems

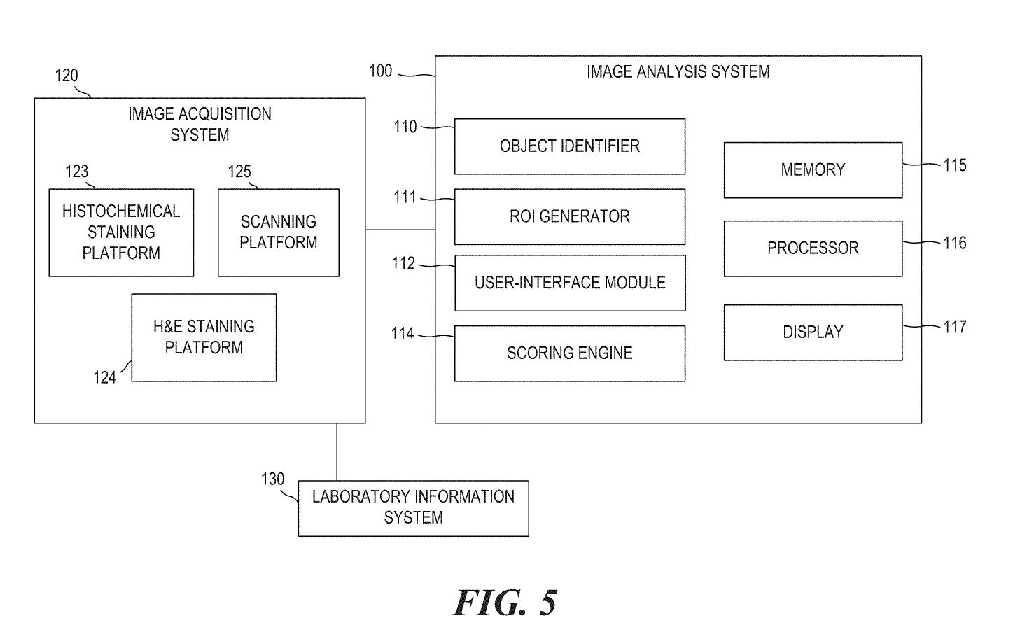

In an embodiment, the present methods are scored manually. In another embodiment, a scoring methodology may be implemented on a scoring system adapted for calculating an HA score from one or more digital images of a tissue section histochemically stained for HA. An exemplary HA scoring system is illustrated at FIG. 5.

The HA scoring system includes an image analysis system 100. Image analysis system 100 may include one or more computing devices such as desktop computers, laptop computers, tablets, smartphones, servers, application-specific computing devices, or any other type(s) of electronic device(s) capable of performing the techniques and operations described herein. In some embodiments, image analysis system 100 may be implemented as a single device. In other embodiments, image analysis system 100 may be implemented as a combination of two or more devices together achieving the various functionalities discussed herein. For example, image analysis system 100 may include one or more server computers and a one or more client computers communicatively coupled to each other via one or more local-area networks and/or wide-area networks such as the Internet.

As illustrated in FIG. 5, image analysis system 100 may include a memory 115, a processor 116, and a display 117. Memory 115 may include any combination of any type of volatile or non-volatile memories, such as random-access memories (RAMs), read-only memories such as an Electrically-Erasable Programmable Read-Only Memory (EEPROM), flash memories, hard drives, solid state drives, optical discs, and the like. For brevity purposes memory 115 is depicted in FIG. 5 as a single device, but it is appreciated that memory 115 can also be distributed across two or more devices.

Processor 116 may include one or more processors of any type, such as central processing units (CPUs), graphics processing units (GPUs), special-purpose signal or image processors, field-programmable gate arrays (FPGAs), tensor processing units (TPUs), and so forth. For brevity purposes processor 116 is depicted in FIG. 5 as a single device, but it is appreciated that processor 116 can also be distributed across any number of devices.

Display 117 may be implemented using any suitable technology, such as LCD, LED, OLED, TFT, Plasma, etc. In some implementations, display 117 may be a touch-sensitive display (a touchscreen).

As illustrated in FIG. 5, image analysis system 100 may also include an object identifier 110, a region of interest (ROI) generator 111, a user-interface module 112, and a scoring engine 114. While these modules are depicted in FIG. 5 as standalone modules, it will be evident to persons having ordinary skill in the art that each module may instead be implemented as a number of sub-modules, and that in some embodiments any two or more modules can be combined into a single module. Furthermore, in some embodiments, system 100 may include additional engines and modules (e.g., input devices, networking and communication modules, etc.) not depicted in FIG. 5 for brevity. Furthermore, in some embodiments, some of the blocks depicted in FIG. 5 may be disabled or omitted. As will be discussed in more detail below, the functionality of some or all modules of system 100 can be implemented in hardware, software, firmware, or as any combination thereof. Exemplary commercially-available software packages useful in implementing modules as disclosed herein include VENTANA VIRTUOSO; Definiens TISSUE STUDIO, DEVELOPER XD, and IMAGE MINER; and Visopharm BIOTOPIX, ONCOTOPIX, and STEREOTOPIX software packages.

After acquiring the image, image analysis system 100 may pass the image to an object identifier 110, which performs a set of computer executable instructions to identify and mark relevant objects and other features within the image that will later be used for scoring. Object identifier 110 may extract from (or generate for) each image a plurality of image features characterizing the various objects in the image as a well as pixels representing expression of the biomarker(s). The extracted image features may include, for example, texture features such as Haralick features, bag-of-words features and the like. The values of the plurality of image features may be combined into a high-dimensional vector, hereinafter referred to as the “feature vector” characterizing the staining pattern of the biomarker. For example, if M features are extracted for each object and/or pixel, each object and/or pixel can be characterized by an M-dimensional feature vector. The output of object identifier 110 is effectively a map of the image annotating the position of objects and pixels of interest and associating those objects and pixels with a feature vector describing the object or pixels.

The features extracted by object identifier 110 may include features or feature vectors sufficient to identify objects in the sample (such as membranes, nuclei, cells, ECM, etc.), and to categorize ECM of the sample as HA-positive, HA-negative, or on the basis of relative intensity. As HA is scored only when localized to tumor-associated ECM, the features extracted by object identifier 110 may include features relevant to identifying: (1) pixels associated with tumor-associated ECM; and/or (2) HA intensity of pixels associated with tumor-associated ECM. In an exemplary embodiment, a feature vector is generated by the object identifier 110 for each pixel, the feature vector including: (1) whether the pixel is associated with ECM; and (2) whether the pixel intensity in the HA stain channel is above or below a threshold level corresponding to background intensity. In another exemplary embodiment, the feature vector generated by the object identifier 110 includes: (1) whether the pixel is associated with tumor-associated ECM; and (2) whether the pixel intensity in the HA stain channel is within a predetermined range selected from: background intensity or below; low (or 1+) intensity; medium (or 2+) intensity; and high (or 3+) intensity. The precise features extracted from the image and incorporated in the feature vector will depend on the type of classification function being applied, and would be well-known to a person of ordinary skill in the art.

The image analysis system 100 may also pass the image to ROI generator 111. The user accesses the ROI generator 111 to identify the ROI or ROIs of the image from which the ECM score will be calculated. In cases where the object identifier 110 is not applied to the whole image, the ROI or ROIs generated by the ROI generator 111 may also be used to define a subset of the image on which object identifier 110 is executed. In an embodiment, the output of the ROI generator 111 is an ROI comprising, consisting essentially of, or consisting of a tumor surface.

In one embodiment, ROI generator 111 may be accessed through user-interface module 112. An image of the HA-stained sample (or a morphologically-stained serial section of the HA-stained sample) is displayed on a graphic user interface of the user interface module 112, and the user annotates one or more region(s) in the image to be considered ROIs. ROI annotation can take a number of forms in this example. For example, the user may manually define the ROI (referred to hereafter as “manual ROI annotation”). In other examples, the ROI generator 111 may assist the user in annotating the ROI (termed, “semi-automated ROI annotation”). For example, the user may delineate one or more regions on the digital image, which the system then automatically transforms into a complete ROI. For example, if the desired ROI is a tumor surface, a user delineates the tumor surface, and the system identifies similar morphological regions by, for example, using computer vision and machine learning, which may then be accepted, rejected, or modified by the user. In other embodiments, ROI generator 111 may automatically suggest an ROI without any direct input from the user (for example, by applying a tissue segmentation function to an unannotated image), which the user may then chose to accept, reject in favor of manual ROI, or edit the ROI and/or append additional ROI areas as deemed appropriate by the user. Other arrangements for annotating ROIs are possible as well, and the scope of the present disclosure should not be considered to limit the manner in which the ROI can be annotated.

In some embodiments, ROI generator 111 may also include a registration function, whereby an ROI annotated in one section of a set of serial sections is automatically transferred to other sections of the set of serial sections. This functionality is especially useful when there are biomarkers being analyzed in conjunction with HA, or when an H&E-stained serial section is provided along with the HA-labeled sections. Thus, for example, an ROI based on morphology may be annotated in an image of an H&E-stained tissue section and the annotated ROI is automatically registered to an HA stained serial section

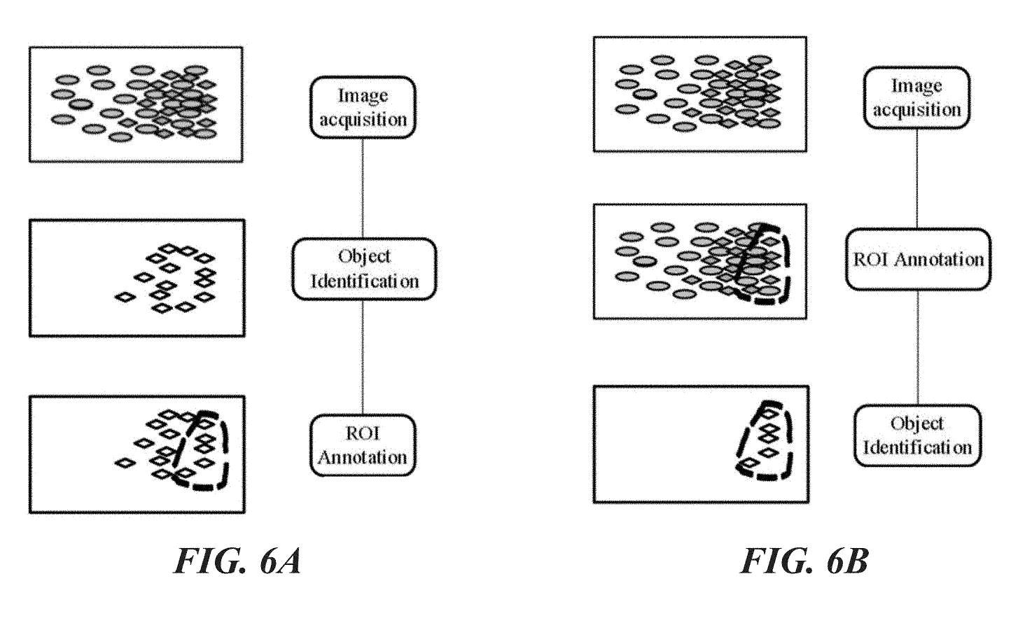

The object identifier 110 and the ROI generator 111 may be implemented in any order. For example, the object identifier 110 may be applied to the entire image first. The positions and features of the identified objects can then be stored and recalled later when the ROI generator 111 is implemented. In such an arrangement, a score can be generated by the scoring engine 114 immediately upon generation of the ROI. Such a workflow is illustrated at FIG. 6A. As can be seen at FIG. 6A, an image is obtained having a mixture of different object (illustrated by dark ovals and dark diamonds). After object identification task is implemented, all diamonds in the image are identified (illustrated by open diamonds). When the ROI is appended to the image (illustrated by the dashed line), only the diamonds located in the ROI region are included in the metric calculation for the ROI. A feature vector is then calculated including the feature metric and any additional metrics used by a scoring function. Alternatively, the ROI generator 111 can be implemented first. In this work flow, the object identifier 110 may be implemented only on the ROI (which minimizes computation time), or it may still be implemented on the whole image (which would allow on-the-fly adjustments without re-running the object identifier 110). Such a workflow is illustrated at FIG. 6B. As can be seen at FIG. 6B, an image is obtained having a mixture of different objects (illustrated by dark ovals and dark diamonds). The ROI is appended to the image (illustrated by the dashed line), but no objects have been marked yet. After object identification task is implemented on the ROI, all diamonds in the ROI are identified (illustrated by open diamonds) and included in the feature metric calculation for the ROI. A feature vector is then calculated including the feature metric(s) and any additional metrics used by the scoring function. It may also be possible to implement the object identifier 110 and ROI generator 111 simultaneously.

After the object identifier 110 and/or ROI generator 111 have been implemented, a scoring engine 114 is implemented. The scoring engine 114 calculates feature metric(s) for the ROI and relevant metrics for objects in the ROI (such as area of HA-positive pixels within tumor-associated ECM, area of tumor-associated ECM with pixels having an HA intensity within one of a plurality of ranges (such as “high,” “medium,” “low” or “1+,” “2+,” or “3+”)). A ROI feature vector including the calculated feature metrics and any other variable derived from the ROI used by the scoring function is compiled and the scoring function (such as a scoring function of Formula 1 or Formula 2) is applied to the ROI feature vector.

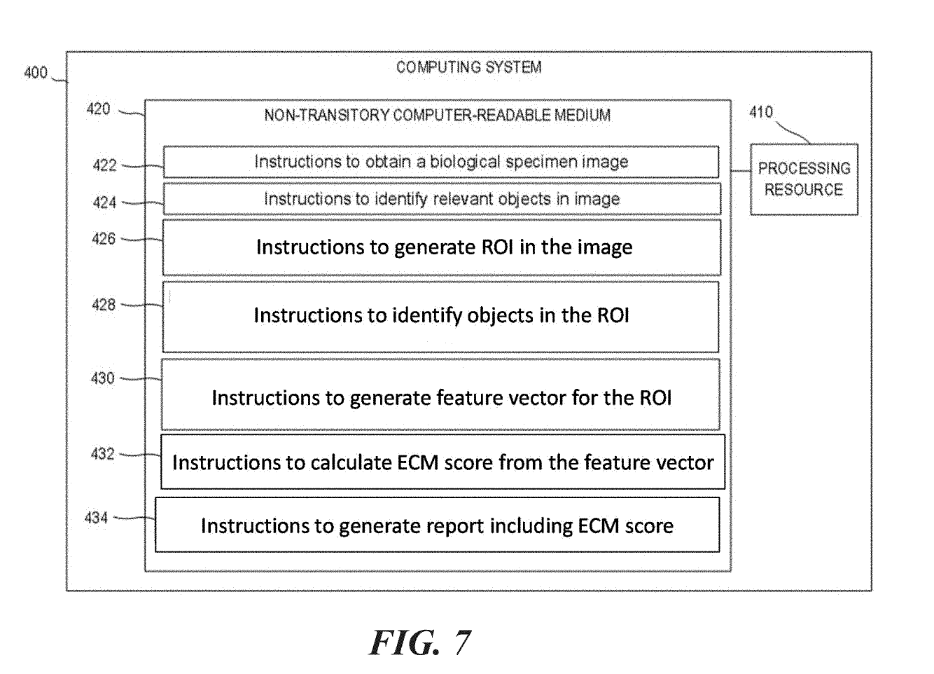

As illustrated in FIG. 7, the image analysis system may include a computing system 400 for implementing the various functions, the computing system 400 comprising a processing resource 410 and a non-transitory computer readable medium 420. The non-transitory computer readable medium 420 includes, for example, instructions to execute function(s) including one or more of: obtain a biological specimen image 422; identify relevant objects in the image 424; generate an ROI in the image 426; identify objects in the ROI useful in identifying ECM space 428; generate a feature vector for the ROI including one or more ROI metrics (such as area of ROI having stain intensity above background or area of ROI having stain intensity within a predefined intensity range (such as digitally measured intensity readings correlating with 1+, 2+, or 3+ intensity scores)) 430; calculate the HA score based on the feature vector 432; and generate a report including the HA score 434.

As depicted in FIG. 5, in some embodiments image analysis system 100 may be communicatively coupled to an image acquisition system 120. Image acquisition system 120 may obtain images of samples and provide those images to image analysis system 100 for analysis and presentation to the user.

Image acquisition system 120 may include a scanning platform 125 such as a slide scanner that can scan the stained slides at 20×, 40×, or other magnifications to produce high resolution whole-slide digital images, including for example slide scanners. At a basic level, the typical slide scanner includes at least: (1) a microscope with lens objectives, (2) a light source (such as halogen, light emitting diode, white light, and/or multispectral light sources, depending on the dye), (3) robotics to move glass slides around (or to move the optics around the slide), (4) one or more digital cameras for image capture, (5) a computer and associated software to control the robotics and to manipulate, manage, and view digital slides. Digital data at a number of different X-Y locations (and in some cases, at multiple Z planes) on the slide are captured by the camera's charge-coupled device (CCD), and the images are joined together to form a composite image of the entire scanned surface. Common methods to accomplish this include:
(1) Tile based scanning, in which the slide stage or the optics are moved in very small increments to capture square image frames, which overlap adjacent squares to a slight degree. The captured squares are then automatically matched to one another to build the composite image; and
(2) Line-based scanning, in which the slide stage moves in a single axis during acquisition to capture a number of composite image “strips.” The image strips can then be matched with one another to form the larger composite image.
   A detailed overview of various scanners (both fluorescent and brightfield) can be found at Farahani et al., Whole slide imaging in pathology: advantages, limitations, and emerging perspectives, Pathology and Laboratory Medicine Int'l, Vol. 7, p. 23-33 (June 2015), the content of which is incorporated by reference in its entirety. Examples of commercially available slide scanners include: 3DHistech PANNORAMIC SCAN II; DigiPath PATHSCOPE; Hamamatsu NANOZOOMER RS, HT, and XR; Huron TISSUESCOPE 4000, 4000XT, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600×; VENTANA ISCAN COREO and ISCAN HT; and Zeiss AXIO SCAN.Z1. Other exemplary systems and features can be found in, for example, WO2011-049608 or in U.S. Published Patent Application No. US 2014-0178169 A1, published Jun. 26, 2014, the content of which is incorporated by reference in its entirety.

Images generated by scanning platform 125 may be transferred to image analysis system 100 or to a server or database accessible by image analysis system 100. In some embodiments, the images may be transferred automatically via one or more local-area networks and/or wide-area networks. In some embodiments, image analysis system 100 may be integrated with or included in scanning platform 125 and/or other modules of image acquisition system 120, in which case the image may be transferred to image analysis system, e.g., through a memory accessible by both platform 125 an system 120. In some embodiments, image acquisition system 120 may not be communicatively coupled to image analysis system 100, in which case the images may be stored on a non-volatile storage medium of any type (e.g., a flash drive) and downloaded from the medium to image analysis system 100 or to a server or database communicatively coupled thereto. In any of the above examples, image analysis system 100 may obtain an image of a biological sample, where the sample may have been affixed to a slide and stained by histochemical staining platform 123, and where the slide may have been scanned by a slide scanner or another type of scanning platform 125. It is appreciated, however, that in other embodiments, below-described techniques may also be applied to images of biological samples acquired and/or stained through other means.

Image acquisition system 120 may also include an automated histochemical staining platform 123, such as an automated IHC/ISH slide stainer. Automated IHC/ISH slide stainers typically include at least: reservoirs of the various reagents used in the staining protocols (including biomarker-specific reagents, detection reagents, wash solutions and other ancillaries), a reagent dispense unit in fluid communication with the reservoirs for dispensing reagent to onto a slide, a waste removal system for removing used reagents and other waste from the slide, and a control system that coordinates the actions of the reagent dispense unit and waste removal system. In addition to performing staining steps, many automated slide stainers can also perform steps ancillary to staining (or are compatible with separate systems that perform such ancillary steps), including: slide baking (for adhering the sample to the slide), dewaxing (also referred to as deparaffinization), antigen retrieval, counterstaining, dehydration and clearing, and coverslipping. Prichard, Overview of Automated Immunohistochemistry, Arch Pathol Lab Med., Vol. 138, pp. 1578-1582 (2014), incorporated herein by reference in its entirety, describes several specific examples of automated IHC/ISH slide stainers and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and Lab Vision Autostainer (Thermo Scientific) automated slide stainers. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. Commercially-available staining units typically operate on one of the following principles: (1) open individual slide staining, in which slides are positioned horizontally and reagents are dispensed as a puddle on the surface of the slide containing a tissue sample (such as implemented on the DAKO AUTOSTAINER Link 48 (Agilent Technologies) and intelliPATH (Biocare Medical) stainers); (2) liquid overlay technology, in which reagents are either covered with or dispensed through an inert fluid layer deposited over the sample (such as implemented on VENTANA BenchMark and DISCOVERY stainers); (3) capillary gap staining, in which the slide surface is placed in proximity to another surface (which may be another slide or a coverplate) to create a narrow gap, through which capillary forces draw up and keep liquid reagents in contact with the samples (such as the staining principles used by DAKO TECHMATE, Leica BOND, and DAKO OMNIS stainers). Some iterations of capillary gap staining do not mix the fluids in the gap (such as on the DAKO TECHMATE and the Leica BOND). In variations of capillary gap staining termed dynamic gap staining, capillary forces are used to apply sample to the slide, and then the parallel surfaces are translated relative to one another to agitate the reagents during incubation to effect reagent mixing (such as the staining principles implemented on DAKO OMNIS slide stainers (Agilent)). In translating gap staining, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap. See WO 2011-139978 A1. It has recently been proposed to use inkjet technology to deposit reagents on slides. See WO 2016-170008 A1. This list of automated histochemical staining platforms is not intended to be comprehensive, and any fully or semi-automated system for performing biomarker-specific histochemical staining may be incorporated into the automated histochemical staining platform 123.

Image acquisition system 120 may also include an automated H&E staining platform 124. Automated systems for performing H&E staining typically operate on one of two staining principles: batch staining (also referred to as “dip 'n dunk”) or individual slide staining Batch stainers generally use vats or baths of reagents in which many slides are immersed at the same time. Individual slide stainers, on the other hand, apply reagent directly to each slide, and no two slides share the same aliquot of reagent. Examples of commercially available H&E stainers include the VENTANA SYMPHONY (individual slide stainer) and VENTANA HE 600 (individual slide stainer) series H&E stainers from Roche; the Dako CoverStainer (batch stainer) from Agilent Technologies; the Leica ST4020 Small Linear Stainer (batch stainer), Leica ST5020 Multistainer (batch stainer), and the Leica ST5010 Autostainer XL series (batch stainer) H&E stainers from Leica Biosystems Nussloch GmbH. H&E staining platform 124 is typically used in workflows in which a morphologically-stained serial section of the biomarker-stained section(s) is desired.

The HA scoring system may further include a laboratory information system (LIS) 130. LIS 130 typically performs one or more functions selected from: recording and tracking processes performed on samples and on slides and images derived from the samples, instructing different components of the HA scoring system to perform specific processes on the samples, slides, and/or images, and track information about specific reagents applied to samples and or slides (such as lot numbers, expiration dates, volumes dispensed, etc.). LIS 130 usually comprises at least a database containing information about samples; labels associated with samples, slides, and/or image files (such as barcodes (including 1-dimensional barcodes and 2-dimensional barcodes), radio frequency identification (RFID) tags, alpha-numeric codes affixed to the sample, and the like); and a communication device that reads the label on the sample or slide and/or communicates information about the slide between the LIS 130 and the other components of the HA scoring system. Thus, for example, a communication device could be placed at each of a sample processing station, automated histochemical stainer 123, H&E staining platform 124, and scanning platform 125. When the sample is initially processed into sections, information about the sample (such as patient ID, sample type, processes to be performed on the section(s)) may be entered into the communication device, and a label is created for each section generated from the sample. At each subsequent station, the label is entered into the communication device (such as by scanning a barcode or RFID tag or by manually entering the alpha-numeric code), and the station electronically communicates with the database to, for example, instruct the station or station operator to perform a specific process on the section and/or to record processes being performed on the section. At scanning platform 125, the scanning platform 125 may also encode each image with a computer-readable label or code that correlates back to the section or sample from which the image is derived, such that when the image is sent to the image analysis system 100, image processing steps to be performed may be sent from the database of LIS 130 to the image analysis system and/or image processing steps performed on the image by image analysis system 100 are recorded by database of LIS 130. Commercially available LIS systems useful in the present methods and systems include, for example, VENTANA Vantage Workflow system (Roche).

### V. Example—Hyaluronan Assay in Pancreatic Ductal Adenocarcinoma (PDA)

Example 1 describes the testing of the scoring assay of the present disclosure.

During the testing of the scoring methods of the present disclosure, multiple tissue samples were used from cell lines and Xenografts (PC3, BxPC3, and DU145), normal human tissue along with tumor tissue samples, e.g., PDA, breast cancer, non-small cell lung cancer (NSCLC), and gastric cancer. Samples were stained on a VENTANA BenchMark automated slide staining system using a TSG6-Rabbit Fc fusion protein (TSG6-Fc1b) as a biomarker-specific reagent and a VENTANA OptiView DAB IHC Detection Kit. Affinity histochemical assays were used to select patients with hyaluronan (HA)-comprising pancreatic ductal adenocarcinoma (PDA) that may benefit from PEGPH20 adjuvant therapy using a cutoff between high HA and low HA of 50% of tumor-associated ECM staining at any HA intensity above background.

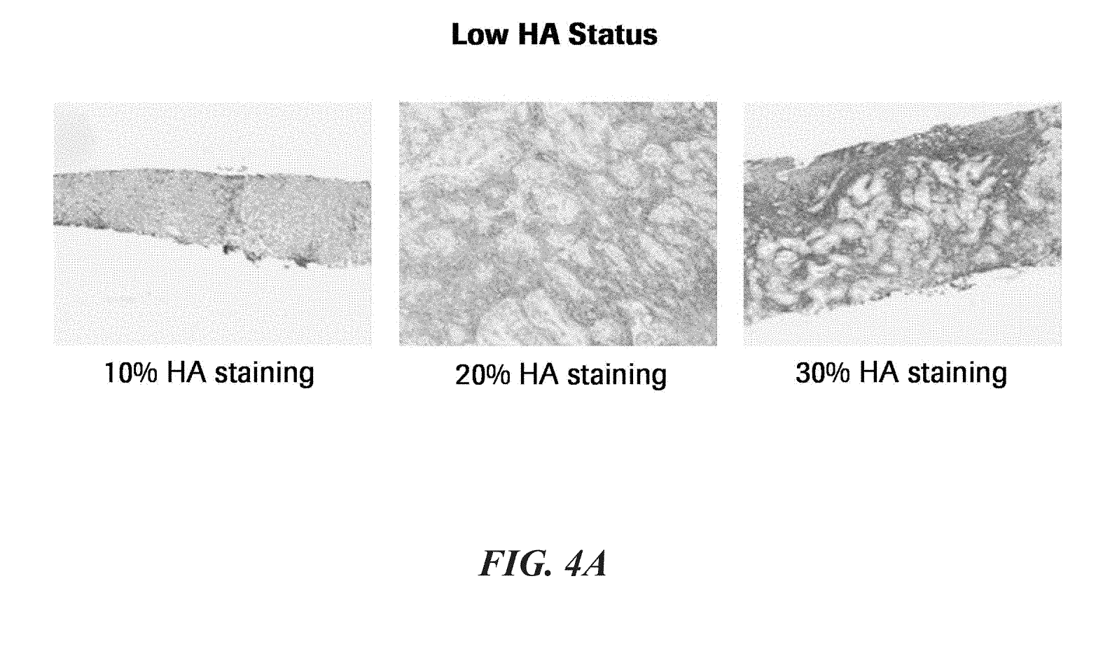

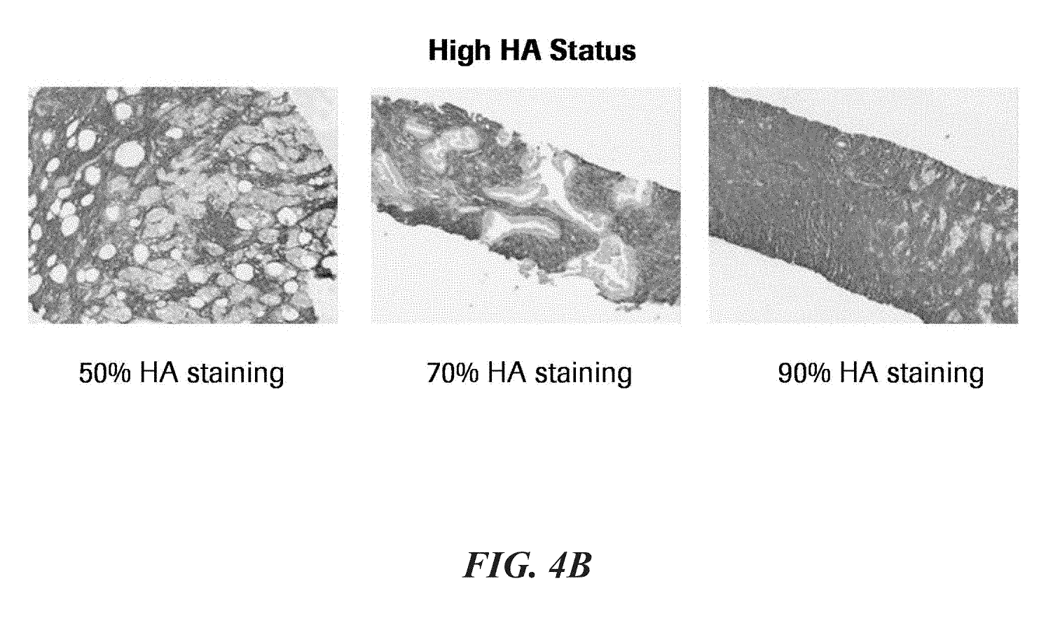

FIG. 4A shows examples of samples scored using the methods of the present disclosure. For example, one sample has a score of 10%, one with 20% and one with 30%. These samples would have a low HA status. FIG. 4B shows additional examples of samples scored using the methods of the present disclosure. For example, one sample has a score of 50%, one with 70% and one with 90%. These samples would have a high HA status.

### VI. Additional Exemplary Embodiments

Exemplary embodiments within the scope of the present disclosure include, but are not limited to, the following:
1. A method of identifying an individual for whom HA treatment is effective by scoring hyaluronan (HA) content in a tissue sample from a tumor of said individual, said method comprising:
   (a) making visible HA in the tissue sample by staining the tissue sample for HA; and
   (b) determining an area of tumor-associated extracellular matrix (ECM) that has HA staining of any intensity over background divided by an area of the entire tumor surface to obtain a percentage to create an HA score, wherein an HA score greater than or equal to a threshold value is designated as high HA, and an HA score of less than the threshold value is designated as low HA, wherein a high HA score is indicative of an individual for whom HA treatment is effective.
2. The method of embodiment 1, wherein the staining of any intensity over background comprises weak (1+) staining for HA, moderate (2+) staining for HA, and strong (3+) staining for HA.
3. The method of embodiment 1, wherein determining the area of the tumor-associated ECM that has HA staining of any intensity over background divided by the area of the entire tumor surface as a percentage comprises
   (a) determining separately (i) the area of the tumor-associated ECM that has weak (1+) staining for HA over the area of the entire tumor surface as a percentage; (ii) the area of the tumor-associated ECM that has moderate (2+) staining for HA over the area of the entire tumor surface as a percentage; and (iii) the area of the tumor-associated ECM that has strong (3+) staining for HA over the area of the entire tumor surface as a percentage; and
   (b) calculating the sum of (i), (ii), and (iii).
4. The method of embodiment 1, wherein the tumor is a tumor of the breast, lung, prostate, pancreas, gastrointestinal tract, or urogenital tract.
5. The method of embodiment 1, wherein the tumor surface comprises tumor cells and associated stroma.
6. The method of embodiment 1, wherein the tumor surface does not comprise necrotic areas.
7. The method of embodiment 6, wherein the non-tumor related stroma is capsule.
8. The method of embodiment 1, wherein background refers to weak or imperceptible non-specific staining that does not interfere with HA reading.
9. The method of embodiment 1, wherein the threshold value is 50%.
10. The method of embodiment 1, wherein the HA treatment comprises PEGPH20.
11. A method of inhibiting growth of a tumor in a patient, said method comprising:
   (a) identifying a patient with a tumor with a high hyaluronan (HA) score as determined by:
      (i) making visible HA in the tissue sample by staining the tissue sample for HA; and
      (ii) determining an area of tumor-associated extracellular matrix (ECM) that has HA staining of any intensity over background divided by an area of the entire tumor surface to obtain a percentage to create an HA score, wherein an HA score greater than or equal to a threshold value is designated as a high HA score; and
   (b) administering an anti-HA treatment to the patient with the tumor with the high HA score, the anti-HA treatment transforms the tumor microenvironment so as to inhibit growth of the tumor.
12. The method of embodiment 11, wherein the anti-HA treatment facilitates access of other treatments to the tumor.
13. The method of embodiment 11, wherein the anti-HA treatment comprises PEGPH20.
14. The method of embodiment 11, wherein the tumor is a tumor of the breast, lung, prostate, pancreas, gastrointestinal tract, or urogenital tract.
15. The method of embodiment 11, wherein the threshold value is 50%.
16. A method of transforming tumor microenvironment of a tumor in a patient, said method comprising:
   (a) identifying a patient with a tumor with a high hyaluronan (HA) score as determined by:
      (i) making visible HA in the tissue sample by staining the tissue sample for HA; and
      (ii) determining the area of tumor-associated extracellular matrix (ECM) that has HA staining of any intensity over background divided by an area of the entire tumor surface to obtain a percentage to create an HA score, wherein an HA score greater than or equal to a threshold value is designated as a high HA score; and
   (b) administering an anti-HA treatment to the patient with the tumor with the high HA score, the anti-HA treatment transforms the tumor microenvironment by depleting HA in the ECM of the tumor.
17. The method of embodiment 16, wherein the anti-HA treatment facilitates access of other treatments to the tumor.
18. The method of embodiment 16, wherein the anti-HA treatment comprises PEGPH20.
19. The method of embodiment 16, wherein the tumor is a tumor of the breast, lung, prostate, pancreas, gastrointestinal tract, or urogenital tract.
20. The method of embodiment 16, wherein the threshold value is 50%.
21. A method of assessing receptiveness of a tumor to a medical treatment, said method comprising:
   (a) making visible an extracellular matrix (ECM) biomarker in a tissue sample of the tumor by staining the tissue sample for the ECM biomarker; and
   (b) determining an area of tumor-associated ECM that has ECM biomarker staining of any intensity over background and dividing the area by an area of the entire tumor surface to obtain an ECM biomarker score;
   wherein an ECM biomarker score greater than or equal to a threshold value is indicative that the tumor is receptive to the medical treatment such that the medical treatment inhibits growth of the tumor, reduces the size of the tumor, or eliminates the tumor.
22. The method of embodiment 21, wherein the ECM biomarker is hyaluronan (HA).
23. The method of embodiment 22, wherein the tumor is a tumor of the breast, lung, prostate, pancreas, gastrointestinal tract, or urogenital tract.
24. A method of determining content of an extracellular matrix (ECM) biomarker in a tumor, said method comprising
   (a) making visible the ECM biomarker in a tissue sample of the tumor by staining the tissue sample of the tumor for the ECM biomarker; and
   (b) determining an area of tumor-associated ECM that has ECM biomarker staining of any intensity over background and dividing the area by an area of the entire tumor surface to obtain an ECM biomarker score;
   wherein an ECM score greater than or equal to a threshold value is indicative that the tumor has a high ECM biomarker content.
25. The method of embodiment 23, wherein the ECM biomarker is hyaluronan (HA).
26. The method of embodiment 24, wherein the tumor is a tumor of the breast, lung, prostate, pancreas, gastrointestinal tract, or urogenital tract.
27. A method comprising:
   (a) annotating a region of interest (ROI) on a digital image of a tissue section histochemically stained for hyaluronan (HA), wherein the ROI comprises a tumor surface;
   (b) detecting in the digital image histochemical staining for HA; and
   (c) determining a HA score according to formula 1 or formula 2:

$$\begin{matrix}HA Score = ( \frac{area ( ECM staining )}{area ( TS )} \times 100 \% ) & Formula 1 \\ HA Score = ( \frac{area ( ECM w / 1 + )}{area ( TS )} \times 100 \% + \frac{area ( ECM w / 2 + )}{area ( TS )} \times 100 \% + \frac{area ( ECM w / 3 + )}{area ( TS )} \times 100 \% ) & Formula 2\end{matrix}$$

wherein:
   area(ECM staining) is an area of tumor-associated ECM having any staining intensity for HA above background staining;
   area(ECM w/1+) is an area of tumor-associated ECM having a HA staining intensity of 1+;
   area(ECM w/2+) is an area of tumor-associated ECM having a HA staining intensity of 2+;
   area(ECM w/3+) is an area of tumor-associated ECM having a HA staining intensity of 3+; and
   area(TS) is the total surface area of tumor surface within the ROI.
28. The method of embodiment 27, wherein HA stain is deposited by contacting the tissue section with a recombinant fusion of TSG6 and rabbit Fc (TSG6-Fc1b) under conditions that facilitate specific binding of TSG6-Fc1b to HA in the tissue section, and reacting the TSG6-Fc1b bound to the tissue section with detection reagents under conditions that result in deposition of a dye in proximity to HA of the tissue section.
29. The method of embodiment 27 or 28, wherein the tissue section is a section of a tumor of the breast, lung, prostate, pancreas, gastrointestinal tract, or urogenital tract.
30. The method of any of embodiments 27-29, wherein the ROI consists of a tumor surface.
31. A system comprising:
   a processor; and
   a memory coupled to the processor, the memory to store computer-executable instructions that, when executed by the processor, cause the processor to perform operations comprising the method of any of embodiments 27-30.
32. The system of embodiment 31, further comprising a scanner or microscope adapted to capture the digital image of the tissue sample and to communicate the image to the computer apparatus.
33. The system of embodiment 31 or 32, further comprising an automated slide stainer programmed to histochemically stain one or more sections of the tissue sample for HA.
34. The system of any of embodiments 31-33, further comprising a laboratory information system (LIS) for tracking sample and image workflow, the LIS comprising a central database configured to receive and store information related to the tissue sample, the information comprising at least one of the following:
   processing steps to be carried out on the tissue section,
   processing steps to be carried out on digital images of tissue sections, and
   processing history of the tissue sections and digital images.
35. A non-transitory computer readable storage medium for storing computer-executable instructions that are executed by a processor to perform operations, the operations comprising the method of any of embodiments 27-30.
36. A system for identifying an individual for whom a hyaluronic acid (HA) directed treatment is effective by scoring a digital image of a tissue sample from a tumor of said individual, said tissue sample having been subjected to affinity histochemical staining for HA, said system comprising:
   an image analysis system comprising a processor and a memory coupled to the processor, the memory to store computer-executable instructions that, when executed by the processor, cause the processor to perform operations comprising:
      identifying within the image a tumor-associated extracellular matrix (ECM) area;
      identifying within the image a total tumor surface area; and
      classifying pixels within the image according to:
         whether or not the pixel is within the tumor-associated ECM area;
         whether or not the pixel has an intensity above the first threshold level, the threshold level being a cutoff between background staining and HA-specific staining; and
         optionally, one of a set of pre-defined ranges in which the HA stain intensity of the pixel falls; and
      applying a set of scoring rules to the image to calculate an HA score, the HA score being a function of an area of tumor-associated ECM having HA staining over background divided by the total tumor surface area, wherein an HA score above a second threshold value is predictive of a response to the HA treatment.
37. The system of embodiment 36, wherein the HA score is calculated according to formula 1:

$$HA Score = ( \frac{area ( ECM )}{area ( TS )} \times 100 \% ) . \qquad Formula 1$$

wherein:
   area(ECM) is the area of the tumor-associated extracellular matrix having HA staining at any intensity above background; and
   area(TS) is the total surface area of the tumor surface.
38. The system of embodiment 36, wherein the set of pre-defined HA stain intensity ranges include (i) a low staining intensity range, (ii) a medium staining intensity range, and (iii) a strong staining intensity range, and wherein the HA score is a function of each of (i), (ii), and (iii) individually divided by the area of the entire tumor surface.
39. The system of embodiment 38, wherein the HA score is calculated according to formula 3:

$$HA Score = ( \frac{area ( ECMlow )}{area ( TS )} + \frac{area ( ECMmed )}{area ( TS )} + \frac{area ( ECMhigh )}{area ( TS )} ) \times 100 \% \qquad Formula 3$$

wherein:
   area(ECMlow) is the area of the tumor-associated extracellular matrix having HA staining intensity within the low staining intensity range;
   area(ECMmed) is the area of the tumor-associated extracellular matrix having HA staining intensity within the medium staining intensity range;
   area(ECMhigh) is the area of the tumor-associated extracellular matrix having HA staining intensity within the strong staining intensity range; and
   area(TS) is the total tumor surface area.
40. The system of embodiment 36, wherein the operations further comprise:
   generating a converted image by converting pixels of the tumor-associated ECM area to one of a plurality of colors on the basis of HA stain intensity of the pixel, wherein pixels having an HA stain intensity falling below the first threshold level have a first color, and pixels having an HA stain intensity falling above the first threshold level have a second color, wherein the first color is different from the second color.
41. The system of embodiment 36, wherein the pixels having the HA stain intensity falling above the first threshold level are classified according to one of the set of pre-defined ranges in which the HA stain intensity of the pixel falls, and wherein the operations further comprise:
   generating a converted image by converting pixels of the tumor-associated ECM area to one of a plurality of colors on the basis of HA stain intensity of the pixel, wherein pixels having an HA stain intensity falling below the first threshold level have a first color, and pixels within each pre-defined range is assigned a color that is different from the first color and different from pixels within a different pre-defined range.
42. The system of any of embodiments 36-41 further comprising an output device communicatively coupled to the image analysis system, wherein the image analysis system is adapted to transmit one or more outputs from the image analysis system, wherein said outputs include at least one of the HA score, the image, and a converted image.
43. The system of any of embodiments 36-42, wherein the system further comprises a scanner adapted to generate the digital image from a tissue section of the tissue sample and to communicate the digital image to the image analysis system or to a non-volatile storage medium.
44. The system of any of embodiments 36-43, wherein the system further comprises an automated IHC/ISH slide stainer and an unstained tissue section of the tissue sample, wherein the automated IHC/ISH slide stainer is adapted to stain the unstained tissue section for HA.
45. The system of embodiment 44, wherein the automated IHC/ISH slide stainer comprises a TSG-6-based probe and a set of specific detection reagents compatible with the TSG-6-based probe.
46. The system of embodiment 45, wherein the TSG-6-based probe is a TSG-6-Fc fusion.
47. The system of embodiment 46, wherein the automated IHC/ISH slide stainer comprises a combination of TSG6-Fc fusion and detection reagents according to Table 1.
48. The system of embodiment 37 or embodiment 39, wherein the second threshold value is 50%.
49. The system of any of embodiments 36-48, wherein the tumor is a tumor of the breast, lung, prostate, pancreas, gastrointestinal tract, or urogenital tract.
50. The system of any of embodiments 36-49, wherein the HA-directed treatment comprises PEGPH20.

Various modifications of the disclosure, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present disclosure, it will be readily apparent to those skilled in the art that modifications may be made thereto which do not exceed the scope of the appended claims. Therefore, the scope of the disclosure is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the disclosures described herein using the phrase “comprising” includes embodiments that could be described as “consisting of”, and as such the written description requirement for claiming one or more embodiments of the present disclosure using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

1. A method of identifying an individual to receive a treatment that comprises enzymatic depletion of hyaluronan (HA), said method comprising:
(a) staining HA in a tissue sample of a tumor from the individual; and
(b) determining an HA score for the tumor by determining a percentage of tumor-associated extracellular matrix (ECM) that has HA staining of any intensity over background divided by an area of the entire tumor surface,
(c) comparing the HA score to a threshold value, and
(d) selecting to patient to receive the treatment if the HA score exceeds the threshold value.

2. The method of claim 1, wherein the staining of any intensity over background comprises weak (1+) staining for HA, moderate (2+) staining for HA, and strong (3+) staining for HA.

3. The method of claim 1, wherein determining the area of the tumor-associated ECM that has HA staining of any intensity over background divided by the area of the entire tumor surface as a percentage comprises
(b1) determining separately (i) the area of the tumor-associated ECM that has weak (1+) staining for HA over the area of the entire tumor surface as a percentage; (ii) the area of the tumor-associated ECM that has moderate (2+) staining for HA over the area of the entire tumor surface as a percentage; and (iii) the area of the tumor-associated ECM that has strong (3+) staining for HA over the area of the entire tumor surface as a percentage; and
(b2) calculating the sum of (i), (ii), and (iii).

4. The method of claim 1, wherein the tumor is a tumor of the breast, lung, prostate, pancreas, gastrointestinal tract, or urogenital tract.

5. The method of claim 1, wherein the tumor surface comprises tumor cells and associated stroma and does not comprise necrotic areas.

6. The method of claim 1, wherein the threshold value is 50%.

7. The method of claim 1, wherein the treatment comprises pegylated hyaluronidase PH20 (PEGPH20).

8. The method of claim 1, further comprising administering the therapy to the patient selected to receive the therapy.

9. The method of claim 1, wherein the HA score is determined by:
annotating a region of interest (ROI) on a digital image of a tissue sample histochemically stained for hyaluronan (HA), wherein the ROI comprises a tumor surface;
detecting in the digital image histochemical staining for HA; and
determining a HA score according to formula 1 or formula 2:
$$\begin{matrix}HA Score = ( \frac{area ( ECM staining )}{area ( TS )} \times 100 \% ) & Formula 1 \\ HA Score = ( \frac{area ( ECM w / 1 + )}{area ( TS )} \times 100 \% + \frac{area ( ECM w / 2 + )}{area ( TS )} \times 100 \% + \frac{area ( ECM w / 3 + )}{area ( TS )} \times 100 \% ) & Formula 2\end{matrix}$$
wherein:
area(ECM staining) is an area of tumor-associated ECM having any staining intensity for HA above background staining;
area(ECM w/1+) is an area of tumor-associated ECM having a HA staining intensity of 1+;
area(ECM w/2+) is an area of tumor-associated ECM having a HA staining intensity of 2+;
area(ECM w/3+) is an area of tumor-associated ECM having a HA staining intensity of 3+; and
area(TS) is the total surface area of tumor surface within the ROI.

10. The method of claim 1, wherein HA is stained by contacting the tissue sample with a recombinant fusion of TSG6 and rabbit Fc (TSG6-Fc1b) under conditions that facilitate specific binding of TSG6-Fc1b to HA in the tissue sample, and reacting the TSG6-Fc1b bound to the tissue section with detection reagents under conditions that result in deposition of a dye in proximity to HA of the tissue section.

11. A system for identifying an individual for whom a hyaluronan (HA) degrading treatment is effective by scoring a digital image of a tissue sample from a tumor of said individual, said tissue sample having been subjected to affinity histochemical staining for HA, said system comprising:
an image analysis system comprising a processor and a memory coupled to the processor, the memory to store computer-executable instructions that, when executed by the processor, cause the processor to perform operations comprising:
identifying within the image a tumor-associated extracellular matrix (ECM) area;
identifying within the image a total tumor surface area; and
classifying pixels within the image according to:
whether or not the pixel is within the tumor-associated ECM area;
whether or not the pixel has an intensity above the first threshold level, the threshold level being a cutoff between background staining and HA-specific staining; and
optionally, one of a set of pre-defined ranges in which the HA stain intensity of the pixel falls; and
applying a set of scoring rules to the image to calculate an HA score, the HA score being a function of an area of tumor-associated ECM having HA staining over background divided by the total tumor surface area, wherein an HA score above a second threshold value is predictive of a response to the HA treatment.

12. The system of claim 11, wherein the HA score is calculated according to formula 1:
$$HA Score = ( \frac{area ( ECM )}{area ( TS )} \times 100 \% ) . \qquad Formula 1$$
wherein:
area(ECM) is the area of the tumor-associated extracellular matrix having HA staining at any intensity above background; and
area(TS) is the total surface area of the tumor surface.

13. The system of claim 11, wherein the set of pre-defined HA stain intensity ranges include (i) a low staining intensity range, (ii) a medium staining intensity range, and (iii) a strong staining intensity range, and wherein the HA score is a function of each of (i), (ii), and (iii) individually divided by the area of the entire tumor surface.

14. The system of claim 13, wherein the HA score is calculated according to formula 3:
$$HA Score = ( \frac{area ( ECMlow )}{area ( TS )} + \frac{area ( ECMmed )}{area ( TS )} + \frac{area ( ECMhigh )}{area ( TS )} ) \times 100 \% \qquad Formula 3$$
wherein:
area(ECMlow) is the area of the tumor-associated extracellular matrix having HA staining intensity within the low staining intensity range;
area(ECMmed) is the area of the tumor-associated extracellular matrix having HA staining intensity within the medium staining intensity range;
area(ECMhigh) is the area of the tumor-associated extracellular matrix having HA staining intensity within the strong staining intensity range; and
area(TS) is the total tumor surface area.

15. The system of claim 11, wherein the operations further comprise:
generating a converted image by converting pixels of the tumor-associated ECM area to one of a plurality of colors on the basis of HA stain intensity of the pixel, wherein pixels having an HA stain intensity falling below the first threshold level have a first color, and pixels having an HA stain intensity falling above the first threshold level have a second color, wherein the first color is different from the second color.

16. The system of claim 11, wherein the pixels having the HA stain intensity falling above the first threshold level are classified according to one of the set of pre-defined ranges in which the HA stain intensity of the pixel falls, and wherein the operations further comprise:
generating a converted image by converting pixels of the tumor-associated ECM area to one of a plurality of colors on the basis of HA stain intensity of the pixel, wherein pixels having an HA stain intensity falling below the first threshold level have a first color, and pixels within each pre-defined range is assigned a color that is different from the first color and different from pixels within a different pre-defined range.

17. The system of claim 11 further comprising an output device communicatively coupled to the image analysis system, wherein the image analysis system is adapted to transmit one or more outputs from the image analysis system, wherein said outputs include at least one of the HA score, the image, and a converted image.

18. The system of claim 11, wherein the system further comprises a scanner adapted to generate the digital image from a tissue section of the tissue sample and to communicate the digital image to the image analysis system or to a non-volatile storage medium.

19. The system of claim 11, wherein the system further comprises an automated IHC/ISH slide stainer and an unstained tissue section of the tissue sample, wherein the automated IHC/ISH slide stainer is adapted to stain the unstained tissue section for HA.

20. The system of claim 19, wherein the automated IHC/ISH slide stainer comprises a TSG-6-Fc fusion and a set of specific detection reagents compatible with the TSG-6-Fc fusion.

21. The system of claim 12, wherein the second threshold value is 50%.

22. The system of claim 14, wherein the second threshold value is 50%.

23. The system of claim 11, wherein the tumor is a tumor of the breast, lung, prostate, pancreas, gastrointestinal tract, or urogenital tract.

24. The system of claim 11, wherein the HA-degrading treatment comprises pegylated hyaluronidase PH20 (PEGPH20).
